(19) United States
(12) Patent Application Publication
(10) Pub. No.: US 2021/0238257 A1
(43) Pub. Date: Aug. 5, 2021

(54) De Novo Formation of the Biliary System by Hepatocyte Transdifferentiation

(71) Applicant: THE REGENTS OF THE UNIVERSITY OF CALIFORNIA, Oakland, CA (US)

(72) Inventors: Holger Willenbring, San Francisco, CA (US); Simone Kurial, San Francisco, CA (US)

(21) Appl. No.: 17/051,089

(22) PCT Filed: Apr. 26, 2019
(86) PCT No.: PCT/US19/29501

§ 371 Date: Oct. 27, 2020

Related U.S. Application Data
(60) Provisional application No. 62/663,675, filed on Apr. 27, 2018.

(51) Int. Cl. C07K 14/71; C12N 15/86; A61P 1/16; A61K 35/407; C12N 15/90; C12N 9/22; C12N 15/11

(52) U.S. Cl. CPC C07K 14/71; C12N 15/86; A61P 1/16; A61K 35/407; C12N 2310/20; C12N 9/22; C12N 15/11; C12N 2750/14043; C12N 2800/80; C12N 15/907

(57) ABSTRACT

The disclosure provides materials and methods useful in forming at least one bile duct or treating cholestatic disease or injury by transdifferentiating hepatocytes to cholangiocytes by delivery of an effective amount of an expressible Transforming Growth Factor β Type I Receptor (TGFBR1), Transforming Growth Factor β Type II Receptor (TGFBR2), SMAD3, SMAD1, SMAD2, SMAD5 or SMAD8/9, in either in vivo or in vitro environments. Another aspect provides a method of forming at least one bile duct or treating a cholestatic disease or injury by delivering an effective amount of JAG1, JAG2, DLL1, DLL3, DLL4, NOTCH1, NOTCH2, NOTCH3, NOTCH4 or the respective NOTCH intracellular domains either in vivo or in vitro. Also provided are methods for correcting mutant alleles of genes in the TGFβ and/or Notch pathways, e.g., JAG1 or NOTCH2, using ZFNs, TALENs, CRISPR or any other genome editing technique. Additionally, methods are provided for inducing increased expression of a normal, or wild-type, allele of a TGFβ or Notch pathway gene such as TGFBR1 or JAG1 using CRISPRa technology. Yet another aspect is drawn to a method of forming at least one bile duct or treating a cholestatic disease or injury by delivering an effective amount of a wild-type hepatocyte or a hepatocyte that has not been engineered to overexpress a gene product.

### FIELD

The field of the disclosure relates to the process of transdifferentiation useful in the treatment of disease. More specifically, the disclosure relates to the treatment of cholestatic disease, for example in humans.

### BACKGROUND

In regenerating organs of adult mammals, differentiated cells can replenish other types of differentiated cells by transdifferentiation, as in the pancreatic islet¹⁰, gastric gland¹¹, lung alveolus¹² and intestinal crypt¹³. Whether mammalian transdifferentiation can build these or other structures de novo is unknown. In the liver, hepatocytes can undergo biliary differentiation to form reactive ductules both in humans¹⁴⁻¹⁷ and animals with cholestatic liver injury^{3-6,18-21} Hepatocyte-derived biliary cells, however, exhibit incomplete biliary and residual hepatocyte differentiation, i.e., the cells are not mature cholangiocytes, and revert back to their original identity after the injury is reversed³⁻⁶. Moreover, hepatocyte-derived ductules do not contribute to bile drainage⁵. These findings are consistent with metaplasia, but not transdifferentiation, and call into question the functional significance of hepatocyte plasticity.

Transforming Growth Factor β Type I Receptor (TGFBR1) is a transmembrane serine/threonine kinase that forms a heteromeric complex with TGFβ type II serine/threonine kinase receptor (TGFBR2), the non-promiscuous receptor for the TGFβ cytokines TGFB1, TGFB2 and TGFB3. This non-promiscuous receptor transduces the TGFB1, TGFB2 and TGFB3 signal from the cell surface to the cytoplasm, thereby regulating a plethora of physiological and pathological processes, including cell cycle arrest in epithelial and hematopoietic cells, control of mesenchymal cell proliferation and differentiation, wound healing, extracellular matrix production, immunosuppression and carcinogenesis. The formation of the receptor complex composed of two TGFBR1 and two TGFBR2 molecules symmetrically bound to the cytokine dimer binding partner results in the phosphorylation and the activation of TGFBR1 by the constitutively active TGFBR2. Activated TGFBR1 phosphorylates SMAD2, which dissociates from the receptor and interacts with SMAD4. The SMAD2-SMAD4 complex is subsequently translocated to the nucleus, where it modulates the transcription of TGFβ-regulated genes. This constitutes the canonical SMAD-dependent TGFβ signaling cascade. The receptor is involved in non-canonical, SMAD-independent TGFβ signaling pathways. For instance, TGFBR1 induces TRAF6 autoubiquitination, which in turn results in MAP3K7 ubiquitination and activation to trigger apoptosis. Also, TGFBR1 regulates the epithelial to mesenchymal transition through a SMAD-independent signaling pathway via PARD6A phosphorylation and activation.

The JAG1 gene encodes the Jagged-1 protein (JAG1), which is a ligand that binds to the receptors NOTCH1, 2, 3 or 4 in the mammalian Notch signaling pathway. Other ligands in this pathway are JAG2 and Delta-like (DLL) 1, 3 and 4. The Notch signaling pathway is a highly conserved pathway that functions to establish and regulate cell fate decisions in many organ systems. Once the JAG1-NOTCH (ligand-receptor) interactions take place, a cascade of proteolytic cleavages is triggered, resulting in transcription activation of downstream target genes. The JAG1 gene is expressed in multiple organ systems in the body and mutations in JAG1 cause the autosomal dominant disorder Alagille syndrome (ALGS).

Despite progress in understanding the processes of tissue and organ regeneration, and despite advances in our knowledge of proteins such as TGFBR1, TGFBR2 and JAG1 that are involved in cell cycle control and cell fate decisions, a need continues to exist in the art for materials, methods and techniques useful in treating conditions or injuries associated with diseased, malformed or traumatized tissues and organs.

### SUMMARY

The disclosure provides materials and methods for treating conditions such as diseases of, or injuries to, mammalian tissues and organs. The disclosed methods rely on transdifferentiation to provide a complete and stable change in cell identity that serves as an alternative to stem-cell-mediated organ regeneration. Transdifferentiation is a form of lineage reprogramming, i.e., a process in which one mature somatic cell transforms into another mature somatic cell. In adult mammals, findings of transdifferentiation have been limited to the replenishment of cells lost from preexisting structures, i.e., in the presence of a fully developed scaffold and niche¹. Disclosed herein is data establishing that transdifferentiation of hepatocytes in the liver can build a structure that failed to form in development—the biliary system in mice that mimic the hepatic phenotype of human Alagille syndrome (ALGS)². In these mice, hepatocytes convert into mature cholangiocytes and form bile ducts that are effective in draining bile and persist after the cholestatic liver injury is reversed, consistent with transdifferentiation. These findings redefine hepatocyte plasticity, which appeared to be limited to metaplasia, i.e., incomplete and transient biliary differentiation as an adaptation to cell injury, based on studies in mice with a fully developed biliary system³⁻⁶. In contrast to bile duct development⁷⁻⁹, demonstrated herein is the independence of de novo bile duct formation by hepatocyte transdifferentiation from NOTCH signaling. TGFβ signaling is identified as the driver and evidence is provided herein in support of this mechanism being active in patients with ALGS. Also shown herein is the ability to target TGFβ signaling in a manner that enhances the formation of the biliary system from hepatocytes, and the data disclosed herein also establish that the transdifferentiation-inducing signals and remodeling capacity of the bile-duct-deficient liver can be harnessed with transplanted hepatocytes. The results disclosed herein define the regenerative potential of mammalian transdifferentiation and reveal opportunities for therapy of ALGS and other cholestatic liver diseases. In some embodiments, a method of treatment of cholestatic disease comprises administration of an effective amount of wild-type hepatocytes, which is supported by data disclosed herein showing that transplanted wild-type hepatocytes form bile ducts in mice lacking bile ducts.

The disclosure contemplates the treatment of any of the following diseases associated with lack of bile ducts. Inherited diseases characterized by an absence of bile ducts include, but are not limited to, biliary atresia, Alagille syndrome, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18 and trisomy 21. Acquired disease exhibiting a lack of bile ducts include, but are not limited to, primary biliary cholangitis, primary sclerosing cholangitis, autoimmune hepatitis, liver transplantation (ischemia, acute or chronic rejection), bone marrow transplantation (chronic graft versus host disease), cancer (Hodgkin lymphoma, Langerhans cell histiocytosis, macrophage activation syndrome), infections (CMV, reovirus type 3, rubella, hepatitis C and B, EBV; Escherichia coli, Cryptosporidium parvum, Treponema pallidum), toxins and drugs, sarcoidosis, and idiopathic adulthood ductopenia.

In one aspect, the invention provides a method of forming a bile duct or ducts comprising introducing at least one expressible coding region in the TGFβ signaling pathway, such as Transforming Growth Factor β Type I Receptor (TGFBR1), Transforming Growth Factor β Type II Receptor (TGFBR2), Mothers Against Decapentaplegic homolog 3 (SMAD3), SMAD1, SMAD2, SMAD5 or SMAD8/9, or in the NOTCH signaling pathway, such as JAG1, JAG2, DLL1, DLL3, DLL4, NOTCH1, NOTCH2, NOTCH3, NOTCH4 or any one or more of the respective NOTCH intracellular domains, into hepatocytes of a patient with a cholestatic disease or injury under conditions where the expression level of the coding region is greater than the wild-type level of expression, thereby inducing transdifferentiation of hepatocytes into mature cholangiocytes that form at least one bile duct. Without wishing to be bound by theory, the method provides a pathway that can compensate for defective signaling, such as defective Notch signaling, by overexpressing TGFBR1, TGFBR2, SMAD3, SMAD1, SMAD2, SMAD5 and SMAD8/9, i.e., the R-SMADs. Alternatively, the method compensates for defective signaling, e.g., Notch signaling, by providing an expressible coding region capable of restoring proper signaling, such as introducing wild-type JAG1, JAG2, DLL1, DLL3, DLL4, NOTCH1, NOTCH2, NOTCH3, NOTCH4 or the respective NOTCH intracellular domains to restore Notch signaling. In some embodiments, the coding region encodes TGFBR1. In some embodiments, the coding region encodes TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains. In some embodiments, the patient has a cholestatic liver injury. In some embodiments, the bile duct contributes to bile drainage. In some embodiments, the bile duct is formed in the absence of Notch signaling. In some embodiments, the TGFBR1 coding region is a constitutive allele, or the TGFBR1 is under the expression control of a constitutive expression control element, e.g., a constitutive promoter, such as the Elongation Factor 1α (EF1α) promoter. In some embodiments, the TGFBR1 coding region is borne by a vector, such as a plasmid, phagemid, cosmid, human artificial chromosome, bacterial artificial chromosome or viral vector. In some embodiments, the viral vector is an Adeno-associated Virus (AAV), such as an Adeno-associated Virus serotype 8 (AAV8).

In some embodiments, the injury, such as a tissue or organ injury, results from a disease, such as human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, cytomegalovirus (CMV) infection, reovirus type 3 infection, rubella infection, hepatitis C infection, hepatitis B infection, Epstein-Barr Virus (EBV) infection; microbe infection, sarcoidosis, or idiopathic adulthood ductopenia. In some embodiments, the disease is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, or trisomy 21. In some embodiments, the disease is human Alagille syndrome (ALGS) wherein the expressible coding region introduced into hepatocytes of the patient encodes JAG1, JAG2, DLL1, DLL3, DLL4, NOTCH1, NOTCH2, NOTCH3, NOTCH4 or any one or more of the respective NOTCH intracellular domains. In some embodiments, the disease is primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, infections (CMV infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the disease is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, or trisomy 21. In some embodiments, the disease is primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, infections (CMV infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the disease is human Alagille syndrome (ALGS), primary biliary cholangitis, or primary sclerosing cholangitis. In some embodiments, the patient has a cholestatic livery injury and practice of a method according to the disclosure results in formation of a bile duct that facilitates redress of the injury.

In some embodiments of the method of treating a cholestatic disease or injury in a patient, the expressible coding region of Transforming Growth Factor β Type I Receptor (TGFBR1), Transforming Growth Factor β Type II Receptor (TGFBR2), SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is introduced into the hepatocytes ex vivo. In some embodiments, the coding region encodes TGFBR1. In some embodiments, the coding region encodes TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains. In some embodiments, the expressible coding region of Transforming Growth Factor β Type I Receptor (TGFBR1), Transforming Growth Factor β Type II Receptor (TGFBR2), SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is introduced into the hepatocytes in vivo. In some embodiments, the coding region encodes TGFBR1. In various embodiments, the nucleic acid comprises a coding region for TGFBR1, TGFBR2, SMAD3, SMAD1, SMAD2, SMAD5 or SMAD8/9, or, to induce Notch signaling, JAG1, JAG2, DLL1, DLL3, DLL4, NOTCH1, NOTCH2, NOTCH3, NOTCH4 or the respective NOTCH intracellular domains.

Another aspect of the disclosure is directed to a method of treating a liver disease or injury in a patient by administering a therapeutically effective amount of a compound that induces, or results in, increased activity of at least one protein effector in hepatocytes of the patient, wherein the protein effector is (a) exogenously administered TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains; (b) an mRNA encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains; or (c) endogenously expressed TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains, wherein the endogenous expression is induced by a small activating RNA. It is understood that when a protein effector itself is administered, the compound inducing, or resulting in, increased protein effector activity is the protein effector itself. In some embodiments, the hepatocyte is a wild-type hepatocyte. In some embodiments, the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the cholestatic disease or injury is human Alagille syndrome. In some embodiments, the hepatocyte is a syngeneic hepatocyte. In some embodiments, the hepatocyte is an autologous hepatocyte.

A related aspect of the disclosure is drawn to a method of inducing an increase in activity of an endogenous effector protein using genome editing technology in the form of activating CRISPR (CRISPRa). Accordingly, the disclosure provides a method of inducing increased expression of an endogenous TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, or NOTCH4 expression control element, or gene, comprising administering a vector comprising a coding region for a guide RNA targeting the expression control region of TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, or NOTCH4 and a coding region for a fusion of deactivated Cas9 (dCas9) and at least one transcriptional activator. In some embodiments, the endogenous coding region, expression control element or gene is a JAG1 or NOTCH2 coding region, expression control element or gene. In some embodiments, the transcriptional activator is VP64, p65, or Rta. In some embodiments, the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the cholestatic disease or injury is human Alagille syndrome.

Yet another aspect of the disclosure provides a method for correcting a mutated endogenous allele of TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, or NOTCH4 expression control element, or gene, comprising administering a vector comprising a coding region for a zinc finger nuclease (ZNF), a coding region for a transcription activator-like effector nuclease (TALEN), or coding regions for clustered regularly interspersed short palindromic repeats (CRISPR)-mediated gene repair, i.e., a guide RNA and Cas9, to a hepatocyte of a patient with a cholestatic disease or injury, thereby correcting the coding region of the mutated endogenous allele to treat the cholestatic disease or injury. In some embodiments, the mutated allele is a mutated JAG1 or a mutated NOTCH2 allele. In some embodiments, the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the cholestatic disease or injury is human Alagille syndrome.

Yet another aspect of the disclosure is drawn to a method of forming at least one bile duct comprising transplanting an effective amount of a hepatocyte not engineered to overexpress a gene product to a patient with a cholestatic disease or injury, thereby inducing formation of at least one bile duct. Without wishing to be bound by theory, this approach provides cells competent for a signaling deficiency underlying a cholestatic liver disease, such as the defective Notch signaling underlying Alagille syndrome. In some embodiments, the hepatocytes are wild-type hepatocytes. In some embodiments, the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the cholestatic disease or injury is human Alagille syndrome, primary biliary cholangitis, or primary sclerosing cholangitis. In some embodiments, the hepatocyte is a syngeneic hepatocyte. In some embodiments, the hepatocyte is an autologous hepatocyte. In some embodiments, the patient has a mutant JAG1 or NOTCH2 allele, wherein the mutant allele is corrected by introducing a vector comprising a zinc finger nuclease (ZFN), a coding region for a transcription activator-like effector nuclease (TALEN), or coding regions for clustered regularly interspersed short palindromic repeats (CRISPR)-mediated gene repair, i.e., a guide RNA and Cas9 into a hepatocyte of a patient with a cholestatic disease or injury, wherein the vector is introduced into the hepatocyte in vitro, thereby correcting the coding region of the mutated endogenous allele to treat the cholestatic disease or injury. In some embodiments, the patient has Alagille syndrome. In some embodiments, the expression of an endogenous normal allele of JAG1 or NOTCH2 is induced by introducing in vitro a vector comprising a coding region for a guide RNA targeting the expression control region of the JAG1 or NOTCH2 allele, and a coding region for a fusion of deactivated Cas9 (dCas9) and at least one transcriptional activator into a hepatocyte of a patient with a cholestatic disease or injury.

Yet another aspect of the disclosure is a method of treating a cholestatic disease or injury comprising administering an effective amount of a hepatocyte not engineered to overexpress a gene product to a patient. In some embodiments, the hepatocyte is a wild-type hepatocyte. In some embodiments, the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia. In some embodiments, the cholestatic disease or injury is human Alagille syndrome, primary biliary cholangitis, or primary sclerosing cholangitis. In some embodiments, the hepatocyte is a syngeneic hepatocyte. In some embodiments, the hepatocyte is an autologous hepatocyte. In some embodiments, the patient has a mutant JAG1 or NOTCH2 allele, wherein the mutant allele is corrected by introducing a vector comprising a zinc finger nuclease (ZFN), a coding region for a transcription activator-like effector nuclease (TALEN), or coding regions for clustered regularly interspersed short palindromic repeats (CRISPR)-mediated gene repair, i.e., a guide RNA and Cas9 into a hepatocyte of a patient with a cholestatic disease or injury, wherein the vector is introduced into the hepatocyte in vitro, thereby correcting the coding region of the mutated endogenous allele to treat the cholestatic disease or injury. In some embodiments, the patient has Alagille syndrome. In some embodiments, the expression of an endogenous normal allele of JAG1 or NOTCH2 is induced by introducing in vitro a vector comprising a coding region for a guide RNA targeting the expression control region of the JAG1 or NOTCH2 allele, and a coding region for a fusion of deactivated Cas9 (dCas9) and at least one transcriptional activator into a hepatocyte of a patient with a cholestatic disease or injury.

Other features and advantages of the present disclosure will become apparent from the following detailed description, including the drawing. It should be understood, however, that the detailed description and the specific examples, while indicating preferred embodiments, are provided for illustration only, because various changes and modifications within the spirit and scope of the invention will become apparent to those skilled in the art from the detailed description.

### BRIEF DESCRIPTION OF THE DRAWINGS

FIG. 1. Hepatocytes can convert into peripheral cholangiocytes and form pBDs contiguous with preexisting hBDs. a, De novo pBD (peripheral bile duct) formation and hepatocyte fate tracing in Alb-Cre^{+/-};Rbpj^{f/f};Hnf6^{f/f};R26ZG^{+/+} mice. Cells identified by dolichos biflorus agglutinin (DBA) lectin labeling and wide-spectrum (ws) cytokeratin (CK) and GFP immunofluorescence (IF); b, IF of hepatocyte-fate-traced P120 Alb-Cre^{+/-}; Rbpj^{f/f};Hnf6^{f/f};R26ZG^{+/+} mouse liver (n=7); c, Biliary tree visualized by retrograde ink injection into the common bile duct of P30 (n=6), P120-P138 (n=6) and ≥P334 (n=6) Alb-Cre^{+/-}; Rbpj^{f/f};Hnf6^{f/f} and P30 (n=3) and P120-P138 (n=5) Rbpj^{f/f};Hnf6^{f/f} mice; d, Maximum projection (top) and 3D reconstruction (bottom) of z-stack image of hepatocyte-fate-traced P120 Alb-Cre^{+/-}; Rbpj^{f/f};Hnf6^{f/f};R26ZG^{+/+} mouse liver (n=2); e, IF and brightfield of hepatocyte-fate-traced P468 Alb-Cre^{+/-};Rbpj^{f/f};Hnf6^{f/f};R26ZG^{+/+} mouse liver after retrograde ink injection into the common bile duct (≥P334, n=3). Scale bars, 2 mm (c, P30, P395 left), 500 μm (c, P138 left), 250 μm (c, P138 right), 100 μm (b, c, P395 right, d, e, left), 25 μm (e, middle). Alb: Albumin; Cre: Cre recombinase; Flp: Flp recombinase; Hnf6: hepatocyte nuclear factor 6, also known as Onecut1 (one cut homeobox 1); Rbpj: recombination signal binding protein for immunoglobulin kappa J region; and R26ZG: Flp reporter.

FIG. 2. Hepatocyte-derived peripheral cholangiocytes are equivalent to normal mature peripheral cholangiocytes. a-c, IF of hepatocyte-fate-traced P120 Alb-Cre^{+/-}; Rbpj^{f/f};Hnf6^{f/f};R26ZG^{+/+} mouse liver (n=3 each). Scale bars, 100 μm (c), 20 μm (a, b). d-g, RNA-seq analysis of normal peripheral cholangiocytes (pC, n=3 mice), hepatocyte-derived peripheral cholangiocytes (HpC, n=4 mice) and RBPJ- and HNF6-deficient hepatocytes (H, n=3 mice). Principal-component analysis (d). Venn diagram showing number of genes significantly differentially up- and down-regulated in pC or HpC versus H (e). Heatmaps of genes reflecting cholangiocyte differentiation, including genes lacking in 3,5-diethoxycarbonyl-1,4-dihydrocollidine (DDC) diet-induced hepatocyte-derived metaplastic biliary cells (top) and other marker genes (bottom) (f). Heatmaps of genes reflecting hepatocyte differentiation, including all differentially expressed cytochrome P450 (CYP) genes enriched in adult mouse liver (top) and other marker genes (bottom) (g). 1-way ANOVA, FDR-corrected P<0.05; fold change >3 (e-g). 2-sided Student's t-test; bold genes P<0.05 for HpC versus pC (f, g).

FIG. 3. HpBD formation entails little proliferation and is driven by TGFβ signaling. a, Possible outcomes, maximum projection image and size distribution of clones in hepatocyte-fate-traced P120 Alb-Cre^{+/-};Rbpj^{f/f};Hnf6^{f/f};R26ZG^{+/+} mice (n=3). b, Possible outcomes, image stack volume projection and size distribution of clones in P150 Alb-Cre^{+/-}; Rbpj^{f/f};Hnf6^{f/f};R26R-Confetti^{+/-} (n=4) and Alb-Cre^{+/-};R26R-Confetti^{+/-} (n=3) mice. c, Ink visualization of biliary tree of >P120 Alb-Cre^{+/-};Rbpj^{f/f};Hnf6^{f/f};Tgfbr2^{f/f} (n=16), Rbpj^{f/f};Hnf6^{f/f};Tgfbr2^{f/f} (n=4) and Alb-Cre^{+/-};Rbpj^{f/f};Hnf6^{f/f} (n=1) mice. d, Sirius-red staining with quantification in >P120 Alb-Cre^{+/-};Rbpj^{f/f};Hnf6^{f/f};Tgfbr2^{f/f} (n=4), Rbpj^{f/f};Hnf6^{f/f};Tgfbr2^{f/f} (n=2) and Alb-Cre^{+/-};Rbpj^{f/f};Hnf6^{f/f} (n=2) mice. Dotted lines represent the means of the indicated P120 mice from FIG. 7f. e, f, Ink visualization of biliary tree and Sirius-red staining with quantification in P100 Alb-Cre^{+/-};Rbpj^{f/f};Hnf6^{f/f} mice that did (n=9) or did not (n=8) receive AAV8-Ef1α-caTgfbr1. Two-sided Student's t-test; *P=0.045. g, Serum total bilirubin in P36 Alb-Cre^{+/-};Rbpj^{f/f};Hnf6^{f/f} mice that did (n=6) or did not (n=8) receive AAV8-Ef1α-caTgfbr1. Two-sided Welch's t-test; *P=0.047. Measure of center is mean (a, b, d, f, g). Scale bars, 2 mm (c), 500 μm (e), 100 μm (d, f), 50 μm (a), 20 μm (b).

FIG. 4. Clinical relevance and therapeutic potential of HpBD formation. a, Experimental design for hepatocyte transplantation. b, IF of P127 mouse (n=5) transplanted with adult GFP-expressing RBPJ- and HNF6-deficient hepatocytes. c, IF of P152 mouse (n=4) transplanted with adult RFP-expressing hepatocytes. d, IF of liver of P72 mouse (n=2) transplanted at P43 with hepatocytes isolated from P287 Alb-Cre^{+/-};R26R-ZsGreen^{+/+} mouse. e, f, Immunohistochemistry and IF of ALGS (n=2) and normal (n=1) human livers. Arrowheads indicate nuclear pSMAD3 in pBDs. Scale bars, 100 μm (b, c, e, f), 50 μm (d).

FIG. 5. Flp-based hepatocyte fate tracing. a, R26ZG allele. b, Experimental design for establishing efficient, specific and constitutive labeling of hepatocytes in normal adult R26ZG^{+/+} mice. c-f, IF of R26ZG^{+/+} mouse liver (n=2) for GFP and the hepatocyte marker major urinary protein (MUP) (c), peripheral and hilar cholangiocyte marker CK19 (d, e), hilar-cholangiocyte-specific marker DBA (e), hepatic stellate cell marker desmin (DES), macrophage marker F4/80 and endothelial cell marker LYVE1 (f) 2 weeks (wks) after intravenous injection of 1×10¹² viral genomes (vgs) of AAV8-Ttr-Flp. g, Reporter activation in R26ZG^{+/+} mice 1 and 2 weeks after intravenous injection of the indicated dose of AAV8-Ttr-Flp (n=1 for each dose and time point). Scale bars, 100 μm.

FIG. 6. Efficiency of hepatocyte fate tracing in mice born with or without pBDs. a, b, Experimental design for hepatocyte fate tracing at P17 and IF at P120 in Rbpj^{f/f};Hnf6^{f/f};R26ZG^{+/+} mice (n=4). c, Correlation of GFP labeling efficiency between hepatocytes and peripheral cholangiocytes in hepatocyte-fate-traced P120 Alb-Cre^{+/-}; Rbpj^{f/f};Hnf6^{f/f};R26ZG^{+/+} mice (n=5, top and bottom). Measure of center is mean. d, e, Experimental design for hepatocyte fate tracing at P39 and IF at P120 in Alb-Cre^{+/-}; Rbpj^{f/f};Hnf6^{f/f};R26ZG^{+/+} mice (n=3). Scale bars, 100 μm.

FIG. 7. HpBDs relieve cholestasis and liver injury. a, Serum total bilirubin levels in P20-29 (n=6), P30-39 (n=33), P40-49 (n=35), P50-59 (n=8), P60-69 (n=46), P70-79 (n=22), P80-89 (n=13), P90-119 (n=20), P120-149 (n=52) and >P150 (n=40) Alb-Cre^{+/-};Rbpj^{f/f};Hnf6^{f/f} and P20-29 (n=5), P30-39 (n=24), P40-49 (n=19), P50-59 (n=7), P60-69 (n=27), P70-79 (n=10), P80-89 (n=11), P90-119 (n=10), P120-149 (n=41) and >P150 (n=25) Rbpj^{f/f};Hnf6^{f/f} mice. 2-sided Welch's t-test; **P=0.0011 at P20-29, ****P=1.7E-13 at P30-39, ****P=8.2E-12 at P40-49, ***P=0.00019 at P50-59, ****P=8.4E-10 at P60-69, ***P=0.00055 at P70-79, ^{ns}P=0.090 at P80-89, ^{ns}P=0.050 at P90-119, ^{ns}P=0.052 at P120-149 and ^{ns}P=0.064 at >P150. b, Serial measurements of serum total bilirubin levels in Alb-Cre^{+/-};Rbpj^{f/f};Hnf6^{f/f} (n=14) and Rbpj^{f/f};Hnf6^{f/f} (n=5) mice. 2-sided Welch's t-test; ^{ns}P=0.11. c-e, Serum ALP, ALT and AST levels in P43-45 (n=13), P69-82 (n=13), P120 (n=14) and P150 (n=11) Alb-Cre^{+/-};Rbpj^{f/f};Hnf6^{f/f} and P43-45 (n=5), P69-82 (n=4), P120 (n=9) and P150 (n=6) Rbpj^{f/f};Hnf6^{f/f} mice. 2-way ANOVA followed by Holm-Sidak multiple comparison test; ****P=0.000058 at P43-45, ****P=0.000019 at P69-82, **P=0.0064 at P120, ^{ns}P=0.22 at P150 and ***P=0.00010 at P120 versus P69-82 (c); **P=0.0050 at P43-45, ^{ns}P=0.47 at P69-82, ^{ns}P=0.30 at P120 and ^{ns}P=0.47 at P150 (d); **P=0.0024 at P43-45, *P=0.015 at P69-82, ****P=0.000073 at P120 and ^{ns}P=0.061 at P150 (e). f, Sirius-red staining in P15 (n=9), P70-90 (n=7) and P120 (n=6) Alb-Cre^{+/-}; Rbpj^{f/f};Hnf6^{f/f} and P15 (n=5), P70-90 (n=3) and P120 (n=3) Rbpj^{f/f};Hnf6^{f/f} mice with quantification. 2-way ANOVA followed by Holm-Sidak multiple comparison test; ^{ns}P=0.94 at P15, ****P=0.000095 at P70-90, **P=0.0074 at P120 and *P=0.027 at P120 versus P70-90. g, Immunohistochemistry and Sirius-red staining in P313 Alb-Cre^{+/-};Rbpj^{f/f};Hnf6^{f/f} mice with persistent or resolved cholestasis (n=1 each). Measure of center is mean (a-f). Scale bars, 100 μm.

FIG. 8. Isolation and gene expression profiling of hepatocyte-derived peripheral cholangiocytes. a, FACS gates for peripheral cholangiocyte (pC, EPCAM⁺DBA⁻) and hilar cholangiocyte (hC, EPCAM⁺DBA⁺) isolation from Alb-Cre^{+/-};Rbpj^{f/f};Hnf6^{f/f} and Rbpj^{f/f};Hnf6^{f/f} mice. b, qPCR analysis of Rbpj floxed genomic DNA in hepatocyte-derived pC (HpC) and hC isolated from Alb-Cre^{+/-};Rbpj^{f/f};Hnf6^{f/f} mice relative to hepatocytes isolated from Rbpj^{f/f};Hnf6^{f/f} mice (n=3 each). Data were normalized to a downstream genomic region of Rbpj to control for gene copy number. Measure of center is mean±SEM. c, d, RNA-seq analysis of normal pC (n=3 mice), HpC (n=4 mice) and RBPJ- and HNF6-deficient hepatocytes (H, n=3 mice). Heatmap of genes reflecting loss of RBPJ and HNF6 (ONECUT1) (c). Rbpj mRNA is present in this knockout mouse as a truncated transcript that does not produce a functional protein²⁶. Heatmap of all differentially expressed CYP genes distinguishing genes associated with mature (M), adolescent (A) and immature (I) hepatocyte differentiation or low expression in the liver (L)²⁹ (d). 1-way ANOVA, FDR-corrected P<0.05; fold change >3 (c, d, except Rbpj and Notch1-4). 2-sided Student's t-test; bold genes P<0.05 for HpC versus pC (c, d).

FIG. 9. Proliferation in HpBDs and reactive ductules. a, Size distribution of wsCK-positive DBA-positive hilar cholangiocyte clones in P90 Alb-Cre^{+/-};Rbpj^{f/f};Hnf6^{f/f};R26R-Confetti^{+/-} (n=2) and Alb-Cre^{+/-};R26R-Confetti^{+/-} (n=2) mouse livers. 2-sided Student's t-test; **P=0.0079 for 3 cells and ***P=0.00092 for 7 cells. b, IF of reactive ductules in hepatocyte-fate-traced P32 Alb-Cre^{+/-};Rbpj^{f/f};Hnf6^{f/f};R26ZG^{+/+} mouse liver (n=3). c, Size distribution of wsCK-positive DBA-negative peripheral cholangiocyte clones in P90 Alb-Cre^{+/-}; Rbpj^{f/f};Hnf6^{f/f};R26R-Confetti^{+/-} (n=2) and Alb-Cre^{+/-};R26R-Confetti^{+/-} (n=2) mouse livers. 2-sided Student's t-test; *P=0.032 for 1 cell, *P=0.024 for 2 cells, *P=0.020 for 3 cells and *P=0.014 for 4 cells. d, IF and breakdown of OPN-positive KI67-positive cells based on CK19 expression in P54 Alb-Cre^{+/-};Rbpj^{f/f};Hnf6^{f/f} mouse liver (n=4). Arrowheads indicate OPN-positive KI67-positive CK19-negative cells. e, IF of liver of >P120 Alb-Cre^{+/-};Rbpj^{f/f};Hnf6^{f/f} and Rbpj^{f/f};Hnf6^{f/f} mice after DDC diet feeding for 2 (n=1 each), 4 (n=3 each) and 6 (n=1 each) weeks (wks). f, IF of liver and breakdown of OPN-positive cells based on hepatocyte fate tracing in >P120 Alb-Cre^{+/-};Rbpj^{f/f};Hnf6^{f/f};R26ZG^{+/+} (n=4) and Rbpj^{f/f};Hnf6^{f/f};R26ZG^{+/+} (n=3) mice fed DDC diet for 5 weeks starting 1 week after hepatocyte fate tracing was induced. Measure of center is mean (a, c, d, f) ±SEM (d, f). Scale bars, 100 μm (b, e, f), 50 μm (d).

FIG. 10. TGFβ signaling in hepatocyte transdifferentiation. a, Ink visualization of biliary tree of P32 Alb-Cre^{+/-};Tgfbr2^{f/f} mouse (n=2). b, IF of P60 Alb-Cre^{+/-};Rbpj^{f/f};Hnf6^{f/f} and Rbpj^{f/f};Hnf6^{f/f} mouse livers (n=2 each). Arrowheads indicate pSMAD3-positive HNF1-positive nuclei. c, Western blot with quantification of pSMAD3 in nuclear extracts from Alb-Cre^{+/-}; Rbpj^{f/f};Hnf6^{f/f}, Rbpj^{f/f};Hnf6^{f/f} and Alb-Cre^{+/-};Rbpj^{f/f};Hnf6^{f/f};Tgfbr2H mouse livers (n=2 each). Source data is shown in FIG. 11. d-f, Experimental design (d) and results of analysis of the effect of TGFβ signaling on biliary differentiation of adult RBPJ- and HNF6-deficient hepatocytes in 3D culture. Phase-contrast images of RPBJ- and HNF6-deficient hepatocyte spheroids embedded in collagen gels and cultured in the presence or absence of the TGFβ inhibitor SB-431542 (SB) for the indicated number of days (d) (e). Relative expression levels of cholangiocyte and hepatocyte genes in freshly isolated hepatocytes and spheroids before and after embedding in collagen gels (f). Gene expression in the liver of a mouse fed choline-deficient ethionine-supplemented (CDE) diet was used as a positive control. Data are from 3 independent cultures per treatment in a representative experiment (n=2). 2-sided Welch's t-test; *P=0.038 for Sox9 5 d, *P=0.044 for Sox9 10 d, **P=0.0034 for Krt19 5 d and **P=0.0071 for Spp 5 d. g, Serum total bilirubin levels in P34-53 Alb-Cre^{+/-};Rbpj^{f/f};Hnf6^{f/f};Tgfbr2^{f/f} (n=16) and Rbpj^{f/f};Hnf6^{f/f};Tgfbr2^{f/f} (n=7) mice. 2-sided Welch's t-test; ****P=0.000024. h, Quantification of Sirius-red staining in P58-100 Rbpj^{f/f};Hnf6^{f/f} mice after intravenous injection of AAV8-Ef1α-caTgfbr1 at P20 (n=4). Gray area represents the range of liver collagen in the indicated Rbpj^{f/f};Hnf6^{f/f} mice from FIG. 7f. Measure of center is mean (c, f, g, h) ±SEM (f). Scale bars, 2 mm (a), 100 μm (e), 50 μm (b), 10 μm (b, inset).

FIG. 11. Western blot source data. Western blots of nuclear lysates from hepatocytes of Rbpj^{f/f};Hnf6^{f/f}, Alb-Cre^{+/-};Rbpj^{f/f};Hnf6^{f/f} and Alb-Cre^{+/-};Rbpj^{f/f};Hnf6^{f/f};Tgfbr2^{f/f} mice. Box highlights section of the blot shown in FIG. 10c. Left panel corresponds to the upper panel of FIG. 10c; right panel corresponds to the lower panel of FIG. 10c. See Brief Description of FIG. 10c.

FIG. 12. Hepatocyte sorting scheme. Hepatocytes were sorted on a FACSAria III using FACSDiva software (BD Biosciences). Hepatocytes were labeled with the indicated antibodies.

FIG. 13. Exogenously administered JAG1 induces transdifferentiation of hepatocytes into cholangiocytes and biliary network expansion in Jag1^{+/-} mice. a, Experimental design for hepatocyte lineage tracing and delivery of AAV8-CMV-Jag1 in mice at postnatal day (P) 2-3. b, Ink visualization of biliary tree at P30 in Jag1^{+/+} mice (n=4) and Jag1^{+/-};R26R^{+/-} mice that did or did not receive AAV8-CMV-Jag1 (n=1 each). c, Ductular reaction derived from lineage-traced hepatocytes at P30 in Jag1^{+/-};R26R^{+/-} mice that did (n=5) or did not (n=3) receive AAV8-CMV-Jag1. d, Quantification of extent of ductular reaction at P30 in Jag1^{+/-};R26R^{+/-} mice that did (n=5) or did not (n=4) receive AAV8-CMV-Jag1. e, Serum levels of the cholestasis marker alkaline phosphatase (ALP) and liver injury markers alanine aminotransferase (ALT) and aspartate transaminase (AST) at P30 in Jag1^{+/-};R26R^{+/-} mice that did (n=5) or did not (n=7) receive AAV8-CMV-Jag1. Untreated control includes Jag1^{+/-}; R26R^{+/-} mice that did not receive AAV8-Ttr-Cre. Scale bars, 1 mm (b), 100 μm (c).

### DETAILED DESCRIPTION

The disclosure provides materials and methods for treating cholestatic disease, which is a group of diseases characterized by impaired flow of bile. Cholestasis is characterized by a decrease in bile flow due to impaired secretion by hepatocytes or to obstruction of bile flow through intra- or extrahepatic bile ducts. Bile is secreted by the liver and facilitates the digestion of fats, and impairments in bile flow can lead to tissue and organ necrosis. Unless treated, cholestatic disease can lead to chronic liver injury and end-stage liver disease.

Provided by the disclosure are methods of using Transforming Growth Factor β Receptor type I (TGFBR1), Transforming Growth Factor β Type II Receptor (TGFBR2), SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains to form at least one bile duct or to treat a cholestatic disease or injury. TGFBR1, an exemplary protein according to the disclosure, forms a heteromeric complex with type II TGFβ Receptor (TGFBR2) when bound to TGFβ, transducing the TGFβ signal from the cell surface to the cytoplasm. TFGβRI is a single-pass type I membrane protein, and the TGFβ receptor protein is a serine/threonine protein kinase. The protein is 503 amino acids (55960 Da) and interacts with CD109. The unphosphorylated protein interacts with peptidyl-prolyl cis-trans isomerase FKBP1A and is stabilized in the inactive conformation. Phosphorylation of the GS region of TFGβRI abolishes FKBP1A binding. When phosphorylated on several residues in the GS region, TFGβRI interacts with SMAD2 (Mothers Against Decapentaplegic homolog 2).

The disclosure also provides a nucleic acid, such as a double-stranded DNA, encoding a biologically active Transforming Growth Factor β Type I Receptor or a fragment, derivative or analog thereof, for delivery to at least one hepatocyte. The accession number for the TGFBR1 gene is NM_004612, and the sequence therein is incorporated herein by reference. The complete polynucleotide sequence of TGFBR1 with the sequence of its chromosomal context on chromosome 9 is provided at GenBank Accession Number NC_000009, incorporated herein by reference. Apparent from a consideration of the disclosure is that nucleic acids encoding a biologically active Transforming Growth Factor β Type II Receptor (Accession Number NM_001024847 for the TGFβR2 polynucleotide encoding variant 1 protein and NM_003242 for the TGFβR2 polynucleotide encoding variant 2 protein), a TGFβ effector coding region, such as a coding region for SMAD3 (Accession Number KR712265 providing the polynucleotide and encoded amino acid sequences), SMAD1 (Accession Number U59423 providing the polynucleotide and encoded amino acid sequences), SMAD2 (Accession Number KJ891567 providing the polynucleotide and encoded amino acid sequences), SMAD5 (Accession Number AH005750 providing the polynucleotide and encoded amino acid sequences) or SMAD8/9 (Accession Numbers NM_001127217, NM_005905, XM_006719827, XM_005266401, XM_005266403, and XM_005266404 providing the polynucleotides of the several variants and the amino acid sequences encoded by each variant), or the NOTCH receptor ligand JAG1 (Accession Number NM_000214 providing the polynucleotide and encoded amino acid sequences), JAG2 (Acc. Nos. NM_002226.5, NM_145159.2), DLL1 (Acc. No. NM_005618.4), DLL3 (Acc. Nos. NM_016941.3, NM_203486.2), DLL4 (Acc. No. NM_019074.4), NOTCH1 (Acc. No. NM_017617.5), NOTCH2 (Acc. Nos. NM_024408.4, NM_001200001.1), NOTCH3 (Acc. No. NM_000435.3), NOTCH4 (Acc. No. NM_004557.4) or the respective NOTCH intracellular domains, as well as coding regions for fragments, derivatives or analogs of any of these biologically active proteins, are expected to be beneficially useful in the methods according to the disclosure.

In addition to providing nucleic acids encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, and fragments, derivatives, and analogs thereof, the disclosure provides nucleic acids comprising at least one expression control element for delivery to hepatocytes. This aspect of the disclosure provides one or more expression control elements to hepatocytes to alter, e.g., increase, the expression of endogenous TGFBRI, TGFBRII, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, or fragments, derivatives, or analogs thereof. In this manner, the expression level and activity of TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is increased in the methods according to the disclosure. Expression control elements contemplated for inclusion in this aspect of the disclosure are heterologous promoters, such as strong promoters, constitutive promoters, inducible promoters, repressible promoters, and any combination thereof known in the art. Other contemplated expression control elements include enhancers, strong ribosome binding sites (e.g., sites conforming more closely to the Kozak consensus sequence for expression in eukaryotic cells), an altered intron sequence or length, stronger intron donor and acceptor splice sites, a polyadenylation sequence, or a transcriptional termination element. Preferred expression control elements for delivery to hepatocytes according to the disclosure are heterologous promoters and enhancers. Also preferred are expression constructs in which at least one expression control element is constitutively active and mutant coding regions expressing a constitutively active protein, such as the D204T mutant TFGβR1.

The polynucleotide (e.g., gene, cDNA, RNA, or fragment, analog or derivative thereof) encoding a Transforming Growth Factor β Type I Receptor (TGFBR1) polypeptide, TGFBR2 polypeptide, SMAD3 polypeptide, SMAD1 polypeptide, SMAD2 polypeptide, SMAD5 polypeptide, SMAD8/9 polypeptide, JAG1 polypeptide, JAG2 polypeptide, DLL1 polypeptide, DLL3 polypeptide, DLL4 polypeptide, NOTCH1 polypeptide, NOTCH2 polypeptide, NOTCH3 polypeptide, NOTCH4 polypeptide or polypeptides of the respective NOTCH intracellular domains can be inserted into an appropriate expression or amplification vector using standard ligation techniques. As used herein, a “vector” is any molecule or moiety that transports, transduces or otherwise acts as a carrier of a polynucleotide according to the disclosure. The vector is typically selected to be functional in the particular host cell employed (i.e., the vector is compatible with the host cell machinery such that amplification of the gene and/or expression of the gene can occur).

Typically, expression vectors used in any of the host cells will contain sequences for plasmid maintenance and cloning and expression of inserted nucleotide sequences. Such sequences, referred to collectively as “flanking sequences”, may include a promoter and other regulatory elements such as an enhancer(s), an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed into a vector, and a selectable marker element. A person skilled in the art will recognize that a target cell may require a specific promoter including, but not limited to, a promoter that is species-specific, inducible, tissue-specific, or cell-cycle-specific. See Parr et al., Nat. Med. 3:1145-9 (1997), the entirety of the contents of which are herein incorporated by reference. Optionally, the vector may contain a “tag” sequence, i.e., an oligonucleotide molecule located at the 5′ or 3′ end of a polypeptide coding sequence of TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains. The oligonucleotide molecule may encode polyHis (such as hexaHis), or another “tag” such as FLAG, HA (hemagglutinin influenza virus) or myc, for which commercially available antibodies exist. This tag is typically fused to the polypeptide upon expression of the polypeptide, and can serve as means for affinity purification of the polypeptide from the host cell. Optionally, the tag can subsequently be removed from a purified polypeptide of TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains by various means such as using certain peptidases.

The flanking sequence may be homologous (i.e., from the same species and/or strain as the host cell), heterologous (i.e., from a species other than the host cell species or strain), hybrid (i.e., a combination of flanking sequences from more than one source), synthetic, or it may be the native nucleic acid flanking sequences of TGFβRI, TGFβRII, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains. The flanking sequences useful in the vectors of this invention may be obtained by any of several methods well known in the art. A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, kanamycin, or G418, (b) complement auxotrophic deficiencies of the cell; or (c) supply critical nutrients not available from the media used for culturing the host cells. The ribosome binding element, commonly called the Shine-Dalgarno sequence (prokaryotes) or the Kozak sequence (eukaryotes), is usually necessary for translational initiation of mRNA. The element is typically located 3′ to the promoter and 5′ to the coding sequence of the polypeptide TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains to be synthesized. Methods used for obtaining each of the elements are well known to the skilled artisan.

Many vectors, e.g., plasmids, cosmids, minicircles, phage, viruses, useful for transferring nucleic acids into target cells are available. The vectors comprising the nucleic acid(s) may be maintained episomally, e.g., as plasmids, minicircle DNAs, viruses such as cytomegalovirus, adenovirus, and adeno-associated virus, or they may be integrated into the target cell genome through homologous recombination or random integration, e.g., retrovirus-derived vectors such as lentivirus, MMLV, HIV-1, and ALV. Vectors may be provided directly to the subject cells by contacting the cells with a vector comprising the polynucleotide of the disclosure such that the vector is taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV). For viral vector delivery, the cells may be contacted with viral particles comprising the polynucleotide of the disclosure.

A preferred vector according to the disclosure is an adeno-associated virus (AAV), which is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac et al., Journal of Translational Medicine 5, 45 (2007). Isolation of the AAV-B 1 serotype is described in Choudhury et al., Mol. Therap. 24(7): 1247-57, 2016. AAV vectors may also comprise self-complementary AAV vectors scAAVs). scAAV vectors contain both DNA strands which anneal together to form double stranded DNA. By skipping second strand synthesis, scAAV s allow for rapid expression in the cell. The sequences of these AAV serotypes are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

In various embodiments, the polynucleotide encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is contained in an adeno-associated virus (AAV) vector. The AAV may be a recombinant AAV virus and may comprise a capsid serotype such as, but not limited to, of AAV1, AAV2, AAV3, AAV4, AAV5, AAV6, AAV7, AAV8, AAV9, AAV9.47, AAV9(hu14), AAV10, AAV11, AAV12, AAV2/5, AAV5/8, AAVrh8, AAVrh10, AAV-DJ, and AAV-DJ8. As a non-limiting example, the capsid or capsids of the recombinant AAV virus is AAV2, AAV5 AAV8, AAV2/5 or AAV5/8.

As noted above, other suitable vectors include retroviruses, for example, lentiviruses, for use in the methods of the disclosure. Commonly used retroviral vectors are defective, i.e., unable to produce viral proteins required for productive infection. Rather, replication of the vector requires growth in a packaging cell line. To generate viral particles comprising genes of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the polynucleotide according to the disclosure into packaging cell lines and of collecting the viral particles that are generated by the packaging lines are well known in the art.

In some embodiments, targeted integration is promoted by the presence of sequences on the polynucleotide of the disclosure that are homologous to sequences flanking the integration site. The “polynucleotide” as recited in the preceding sentence means the polynucleotide encoding a TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, or a fragment, a derivative, or an analog thereof, or an expression control element as disclosed herein. For example, targeted integration using the polynucleotides described herein may be achieved following conventional transfection techniques, e.g., techniques used to create gene knockouts or knock-ins by homologous recombination. In other embodiments, targeted integration is promoted both by the presence of sequences on the polynucleotide of the disclosure that are homologous to sequences flanking the integration site, and by contacting the cells with the polynucleotide described herein in the presence of a site-specific recombinase, such as a recombinase from the Integrase or Resolvase families.

The disclosure provides methods for administering or delivering the nucleic acids encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, or fragments, derivatives or analogs thereof, to individuals, e.g., humans that have a cholestatic disease. A variety of cholestatic diseases are amenable to treatment according to the methods of the disclosure. In general, any cholestatic disease is treatable by the methods disclosed herein. In particular, the disclosure contemplates treatment of human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, cytomegalovirus (CMV) infection, reovirus type 3 infection, rubella infection, hepatitis C infection, hepatitis B infection, Epstein-Barr virus infection; microbe infection, sarcoidosis, or idiopathic adulthood ductopenia. Any known method of administering or delivering the nucleic acids encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, or fragments, derivatives or analogs thereof is contemplated. In some embodiments, the nucleic acids, e.g., an AAV vector comprising a polynucleotide encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is administered by injection into a peripheral vein. In some embodiments, the nucleic acid is targeted to one or more particular tissues or organs, such as targeting the liver by injection of the vector comprising the nucleic acid encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, or fragments, derivatives or analogs thereof, into the portal vein, hepatic artery or spleen. In addition to providing the encoding nucleic acid as a DNA polynucleotide in a suitable vector, the disclosure contemplates delivery of an mRNA encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, or fragments, derivatives or analogs thereof, to treat cholestatic disease.

As an alternative to using viral vector to deliver the nucleic acids encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains, synthetic mRNAs, small activating RNAs, or recombinant proteins can be used to provide these therapeutic factors in vivo. In addition, small molecules can be used to activate the TGFβ or NOTCH signaling pathways in vivo. Intravenously injected in vitro-synthesized mRNAs encapsulated in lipid nanoparticles are readily taken up by hepatocytes through receptor-mediated endocytosis, leading to rapid delivery of the mRNA to the cytoplasm and production of the therapeutic protein. Small activating RNAs are short double-stranded oligonucleotides that can be targeted to a gene's promoter to induce transcriptional elongation. Formulation in liposomal nanoparticles facilitates efficient uptake and target gene activation in hepatocytes after intravenous injection. Recombinant proteins, like Fc chimeric fusion proteins composed of an immunoglobulin Fc domain that is directly linked to another peptide, are effective in the liver after intravenous injection; they are particularly useful to provide ligands such as JAG1, JAG2, DLL1, DLL3 or DLL4. Small molecules activating TGFβ or NOTCH signaling exist and can be targeted to exert their function specifically in the liver after intravenous injection. Because of low immunogenicity, mRNAs, small activating RNAs, recombinant proteins, and small molecules can be repeatedly applied, thereby overcoming their transient effect.

As an alternative to delivering the nucleic acids encoding JAG1 or NOTCH2, the mutated JAG1 or NOTCH2 allele can be corrected in situ using genome editing technology, such as, but not limited to, zinc finger nucleases (ZNFs), transcription activator-like effector nucleases (TALENs) or clustered regularly interspaced short palindromic repeats (CRISPR). It is known in the art that ZNFs and the Transcription Activator-Like (TAL) component of TALENs are amenable to genetic engineering to tailor the binding site of the ZNF or TALEN to a DNA site of interest, and it is further known that these components can be fused to non-specific nucleases such as the FokI nuclease cleavage domain to yield nucleases targeting a DNA site of interest for editing. Analogously, CRISPR has been developed to the point where the editing of a given region of DNA, such as a gene, coding region or expression control element, is known in the art. Each of these genome editing techniques is known in the art to be useful in correcting mutations in DNA, including mutations in coding regions, expression control elements or, more generally in genes or intergenic DNA. Further, each of these techniques is amenable to administration to hepatocytes in vivo in patients with cholestatic injury or disease and to introduction into hepatocytes from such patients in vitro, suitable for in vitro amplification of the hepatocytes and/or re-introduction of such cells into the patients.

In addition, the expression level of a normal or non-mutated allele such as the non-mutated JAG1 or NOTCH2 allele can be induced using activating CRISPR (CRISPRa). To induce the expression of a normal, e.g., wild-type, allele of JAG1 or NOTCH2, in a hepatocyte of a patient with a cholestatic disease or injury, a vector is administered to the hepatocyte(s) that comprises a deactivated Cas9 (dCas9), i.e., a Cas9 coding region in which the nuclease domains have been inactivated by mutation. The dCas9 is fused to one or more transcriptional activators, e.g., VP64, p65 and Rta. The vector further comprises a guide RNA that targets upstream of the transcriptional start site (TSS) of the target gene, such as the normal, or wild-type, allele of JAG1 or NOTCH2 in a patient exhibiting the haploinsufficiency characteristic of Alagille syndrome. The administration of such a vector results in induced expression of the normal allele such as the normal JAG1 or NOTCH2 allele in hepatocytes of patients with a cholestatic disease or injury. As with the gene correction methodologies noted above, induction of endogenous gene expression, e.g., JAG1 or NOTCH2 expression, using CRISPRa is amenable to in vivo administration of the vector or introduction of the vector into hepatocytes from such patient in vitro, followed by amplification and/or re-introduction of such cells into the patients. These gene-editing tools can be efficiently delivered to hepatocytes in vivo using viral vectors, particularly AAV vectors.

Because Alagille syndrome is caused by haploinsufficiency of JAG1, i.e., patients have a mutated and a normal allele, CRISPR-mediated activation (CRISPRa) can be used to increase expression of the normal allele. The components of CRISPRa can be efficiently delivered to hepatocytes in vivo using viral vectors, particularly AAV vectors.

The following examples illustrate the subject matter of the disclosure and are not intended to limit the scope of the disclosure. Example 1 provides the methods used in the experiments disclosed herein, Example 2 describes the mouse model, Example 3 discloses the tracing of peripheral bile duct formation, Example 4 shows that the peripheral bile ducts that were formed were contiguous with the extrahepatic biliary system, Example 5 discloses the examination of cell identity in the transdifferentiation of hepatocytes to cholangiocytes, Example 6 relates the determination of the relative numbers of transdifferentiating hepatocytes, Example 7 shows the results of assessing proliferation of transdifferentiated hepatocytes in a mouse model of cholestatic liver injury induced by DDC, and Example 8 discloses the assessment of the role played by TGFβ signaling in transdifferentiated hepatocyte formation of bile ducts.

### Example 1

### Methods

### Mice

Alb-Cre^{+/-};Rbpj^{f/f};Hnf6^{f/f} mice (mixed background) were previously reported^{2,26}. R26R-RFP^{+/+39} (C57BL/6) and R26NZG^{+/+40} (FVB) mice were used. Flp-reporter mice were generated by crossing R26NZG^{+/+} mice with EIIa-Cre^{+/+41} mice (C57BL/6) to remove the Cre-reporter element and then crossing out the EIIa-Cre. These R26ZG^{+/+} mice were crossed with Alb-Cre^{+/-}; Rbpj^{f/f};Hnf6^{f/f} mice to generate Alb-Cre^{+/-};Rbpj^{f/f};Hnf6^{f/f};R26ZG^{+/+} mice. R26R-Confetti^{+/+42} mice (C57BL/6) were crossed with Alb-Cre^{+/-};Rbpj^{f/f};Hnf6^{f/f} mice to generate Alb-Cre^{+/-}; Rbpj^{f/f};Hnf6^{f/f};R26R-Confetti^{+/-} mice. Tgfbr2^{f/f43} mice (C57BL/6) were crossed with Alb-Cre^{+/-}; Rbpj^{f/f};Hnf6^{f/f} mice to generate Alb-Cre^{+/-};Rbpj^{f/f};Hnf6^{f/f};Tgfbr2^{f/f} mice. Because Tgfbr2 (68.39 cM) and Hnf6 (41.93 cM) are both on chromosome 9, recombinants were generated at 0.1356 (35/258 mice) observed frequency (0.26 expected frequency). Different founder recombinants were intercrossed to generate Alb-Cre^{+/-};Rbpj^{f/f};Hnf6^{f/f};Tgfbr2^{f/f} mice. R26R-ZsGreen^{+/+44} mice (C57BL/6) were crossed with Alb-Cre^{+/-} mice to generate Alb-Cre^{+/-};R26R-ZsGreen^{+/+} mice. Alb-Cre^{+/-};Rbpj^{f/f};Hnf6^{f/f} mice were crossed with Rag2^{-/-45};Il2rg^{-/-46} mice (mixed background) to generate Alb-Cre^{+/-};Rbpj^{f/f};Hnf6^{f/f};Rag2^{-/-};Il2rg^{-/-} mice. Jag1^{+/-55} (C57BL/6) were bred to R26R-RFP^{+/+} mice to generate Jag1^{+/-};R26R-RFP^{+/-} mice. Male and female mice of the indicated age and genotype were chosen randomly for inclusion in experiments. The mouse used as a positive control for biliary gene expression was a C57BL/6 wild-type mouse fed choline-deficient diet (MP Biomedicals) and given 0.15% (w/v) ethionine (Sigma-Aldrich) in the drinking water (CDE diet) for 3 weeks. All mice were kept under barrier conditions. All procedures were approved by the Institutional Animal Care and Use Committee at UCSF or CCHMC.

### Adeno-Associated Virus

The AAV-Ttr-Flp plasmid was generated by removing Cre from AAV-Ttr-Cre⁴⁷ and replacing it with Flpo⁴⁸ from pPGKFlpobpA (Addgene 13793); viruses were produced by Vector Biolabs and used at a high dose of 1-3×10¹² vgs (viral genomes) or low dose of 4×10¹¹ vgs. The AAV-Ef1α-caTgfbr1 plasmid was built by VectorBuilder to contain the activating T204D⁴⁹ mutation; virus was produced by Vector Biolabs and used at a dose of 1×10¹¹ vgs. Titers were determined using qPCR. Viruses were delivered by tail vein injection in volumes ≤100 μL to prevent hydrodynamic effects. AAV-CMV-Jag1 plasmid was built by VectorBuilder; virus was produced by Vector Biolabs and used at a dose of 1×10¹¹ vgs.

### Tissue Collection

Livers from Alb-Cre^{+/-};Rbpj^{f/f};Hnf6^{f/f};R26ZG^{+/+}, R26R-Confetti^{+/-} and transplanted mice were perfused with ice-cold PBS followed by 4% paraformaldehyde (PFA). Samples were cut into slices and fixed overnight in 4% PFA at 4° C. Livers from R26R-RFP^{+/+}, Alb-Cre^{+/-}; Rbpj^{f/f};Hnf6^{f/f}, Rbpj^{f/f};Hnf6^{f/f} and Alb-Cre^{+/-};Rbpj^{f/f};Hnf6^{f/f};R26ZG^{+/+} mice were fixed overnight in 4% PFA at 4° C. For thin sectioning, samples were moved to 30% sucrose overnight at 4° C. to cryopreserve and then embedded in OCT (Tissue-Tek) or processed for paraffin embedding and sectioning. A Leica CM3050 S cryostat was used to cut 6 μm cryosections for staining and imaging. For 3D and sparse-labeling clonal analysis, liver samples were embedded in 4% agarose and sectioned on a Leica VT100 S vibratome. For clonal analysis in R26R-Confetti^{+/-} mice, liver samples were cut into 1 mm slivers, immunostained, washed in PBS, equilibrated in 30% sucrose and embedded in OCT. The 1 mm slivers were then cryosectioned at 100 μm, stained with Hoechst and mounted in PBS for confocal imaging. For analysis of Flp-based hepatocyte fate tracing in R26ZG^{+/+} mice, liver samples were fixed in neutral-buffered formalin containing zinc (Z-Fix, Anatech), embedded in paraffin and sectioned to 5 μm.

### Human Liver Tissue

Explant samples from the regenerative nodule and nonregenerated liver tissue of a 3-year-old male patient with ALGS were previously described; regenerative nodule and nonregenerated liver tissue contained the same heterozygous JAG1 exon 1-26 deletion³⁶. Samples were obtained with patient consent and approval from the Commission Cantonale d'Ethique de la Recherch CCER. Biopsy samples from the regenerative nodule and nonregenerated liver tissue of a 15-year-old male patient with ALGS caused by a heterozygous c.499T>A (p.W167R) JAG1 mutation and resection samples from a histologically normal region of the liver of a 35-year-old male undergoing surgery for metastasis of rectal adenocarcinoma were obtained with patient consent and approval from the UCSF Institutional Review Board.

### Immunostaining and Histology

Cryosections were blocked in 10% normal serum and permeabilized in 0.3% Triton-X before staining with primary and secondary antibodies listed in Tables 2 and 3, respectively. Paraffin-embedded samples were deparaffinized and underwent antigen retrieval in sodium citrate buffer (Bio-Genex or Vector Labs) or Tris EDTA buffer (10 mM Tris Base, 1 mM EDTA, 0.05% Tween 20, pH 9.0) in a steamer or pressure cooker for 15 minutes before blocking and permeabilization. Thin sections were mounted in FluorSave (MilliporeSigma) or 50% glycerol. Vibratome sections were stained free-floating in 12-well dishes and cleared in Focus Clear (Cedarlane Laboratories) before being mounted in Mount Clear (Cedarlane Laboratories) for confocal imaging. All samples stained with anti-wsCK antibody underwent antigen retrieval in 100 mM Tris buffer (pH 9.5) at 55° C. for 2 hours before blocking. Staining with anti-pSMAD3 antibody used a biotinylated secondary antibody followed by avidin/biotin-based peroxidase and tyramide amplification. Staining with anti-SSTR2 antibody used an HRP secondary antibody followed by tyramide-based amplification. M.O.M. Kit (Vector Laboratories) was used for antibodies raised in mice. DAPI or Hoechst was used to stain nuclear DNA. Chromogenic detection used an HRP secondary antibody followed by avidin/biotin-based peroxidase amplification and DAB substrate exposure. Sirius-red staining was performed on deparaffinized and rehydrated samples using a 0.1% Picrosirius-red solution (Dudley Corporation and Newcomer Supply) and 0.5% acetic acid water washes or was done at Peninsula Histopathology Laboratory.

TABLE 2
Primary Antibodies.

| Antigen | Dilution | Host | Supplier | Catalog # | Clone/Lot # |
|---|---|---|---|---|---|
| ACTIN | 1:500 | Mouse | Seven | LMAB- | Clone C4 |
| | | | Hill | C4 | |
| | | | Bio- | | |
| | | | reagents | | |
| acTUB | 1:100 | Mouse | Abcam | ab24610 | Clone 6-11B-1 |
| CD11b- | 1:100 | Rat | Tonbo | 60-0112 | C0112012616602, |
| PE-Cy7 | | | Bio | | C0112012616603 |
| CD31- | 1:100 | Rat | Bio- | 102417 | B212261, |
| PE-Cy7 | | | Legend | | B212262 |
| CD45- | 1:100 | Rat | Tonbo | 60-0451 | C0451080615602, |
| PE-Cy | | | Bio | | C0451080615603 |
| CK7 | 1:25 | Rabbit | Ventana | 790-4462 | Clone SP52 |
| CK19 | 1:100 | Rabbit | Abbomax | 602-670 | 1101-766 |
| CK19 | 1:50 | Rat | DSHB | Troma- | 8G4, 48 |
| | | | | III- [symbol] | |
| DBA- | 1:100- | | Vector | B-1035 | ZA0417 |
| biotin | 300 | | Labs | | |
| DBA- | 1:80 | | Vector | FL-1031-2 | ZA0722 |
| FITC | | | Labs | | |
| DESMIN | 1:100 | Rabbit | Thermo | RB9014P0 | 9014P1604F, |
| | | | Fisher | | 9014P1604G |
| | | | Scientific | | |
| EPCAM | 1:100- | Rat | BD | 552370 | 7089730 |
| | 500 | | Bio- | | |
| | | | sciences | | |
| EPCAM- | 1:100 | Rat | Bio- | 118225 | B204922 |
| BV421 | | | Legend | | |
| F4/80 | 1:100 | Rat | BioRad | MCA497 | Clone A3-1, |
| | | | | GA | Batch 1014, 1610 |
| GFP | 1:200- | Chicken | Abcam | ab13970 | GR236651-10, |
| | 500 | | | | GR236651-21 |
| GFP | 1:500 | Rabbit | Abcam | ab6556 | GR277888-1 |
| HNF1 | 1:50 | Rabbit | Santa | SC-8986 | G3112 |
| | | | Cruz | | |
| KI67 | 1:200 | Rabbit | Thermo | RM-9106- | Clone SP6, Lot |
| | | | Fisher | S0 | 9106S1607G |
| | | | Scientific | | |
| LYVE1 | 1:200 | Rabbit | Relis- | 103- | 1208R1, |
| | | | Tech | PA50AG | 1210R03 |
| MUP | 1:250 | Goat | Cedar- | GAM/MUP | 5023 |
| | | | lane | | |
| OC2- | 1:20 | Rat | Craig | [symbol] | [symbol] |
| 2F8 | | | Dorrell | | |
| OPN | 1:250 | Goat | R & D | AF808 | BDO0613101, |
| | | | Systems | | BDO0617041 |
| pSMAD3 | 1:400- | Rabbit | Abcam | ab52903 | GR128879-61 |
| | 500 | | | | |
| | (IF), | | | | |
| | 1:1,000 | | | | |
| | (WB) | | | | |
| pSMAD3 | 1:400 | Rabbit | Cell | 9520 | Clone C25A9 |
| | | | Signaling | | |
| SOX9 | 1:500 | Rabbit | Millipore | AB5535 | 2847051, |
| | | | Sigma | | 2975230 |
| SSTR2 | 1:500 | Rabbit | Bioss | bs-1138R | 9A25V1 |
| wsCK | 1:200 | Rabbit | Dako | Z0622 | 10088577 |

55 Huch, M. et al. In vitro expansion of single Lgr5+ liver stem cells induced by Wnt-driven regeneration. Nature 494, 247-250 (2013). [symbol]
indicates data missing or illegible when filed

TABLE 3
Secondary Antibodies.

| Reactivity | Species | Fluorochrome | Dilution | Supplier | Catalog # | Lot # |
|---|---|---|---|---|---|---|
| Chicken | Donkey | Alexa Fluor | 1:500 | Jackson Immuno | 703-525-155 | 116967, |
| | | 488 | | | | 113085 |
| Goat | Donkey | Alexa Fluor | 1:500 | Thermo Fisher | A11055 | 1869589 |
| | | 488 | | Scientific | | |
| Goat | Donkey | Alexa Fluor | 1:500 | Jackson Immuno | 705-585-147 | 106994 |
| | | 594 | | | | |
| Goat | Donkey | Alexa Fluor | 1:500 | Thermo Fisher | A11058 | 1445994 |
| | | 594 | | Scientific | | |
| Goat | Donkey | Alexa Fluor | 1:200 | Thermo Fisher | A21447 | 1841382, |
| | | 647 | | Scientific | | 1661244 |
| Goat | Donkey | Biotin | 1:1,000 | Jackson Immuno | 705-065-003 | 77098 |
| Goat | Donkey | Cy3 | 1:500 | Jackson Immuno | 705-065-147 | 114067 |
| Mouse | Horse | HRP | 1:1,000 | Cell Signaling | 7076 | 27 |
| Rabbit | Goat | Alexa Fluor | 1:300 | Jackson Immuno | 111-545-144 | 105878 |
| | | 488 | | | | |
| Rabbit | Donkey | Alexa Fluor | 1:200-500 | Thermo Fisher | A31572 | 1917920 |
| | | 555 | | Scientific | | |
| Rabbit | Donkey | Alexa Fluor | 1:200 | Jackson Immuno | 711-605-132 | 118217 |
| | | 647 | | | | |
| Rabbit | Donkey | Biotin | 1:1,000 | Jackson Immuno | 711-065-152 | 128874 |
| Rabbit | Donkey | Cy3 | 1:500 | Jackson Immuno | 711-165-152 | 117211, |
| | | | | | | 105494 |
| Rabbit | Goat | HRP | 1:1,000 | Cell Signaling | 7074 | 26 |
| Rat | Goat | HRP | 1:500 | Jackson Immuno | 111-035-144 | 91036 |
| Rat | Donkey | Alexa Fluor | 1:500 | Thermo Fisher | A21208 | 1789917 |
| | | 488 | | Scientific | | |
| Rat | Donkey | Alexa Fluor | 1:200 | Jackson Immuno | 712-605-153 | 123845, |
| | | 647 | | | | 110001, |
| | | | | | | 127806 |
| Rat | Donkey | Cy2 | 1:300 | Jackson Immuno | 712-225-150 | 102982 |
| Rat | Goat | PE | 1:200 | HD Biosciences | 550767 | 5107876 |
| Strepravidin | | Alexa Fluor | 1:300 | Thermo Fisher | S-11227 | 1704463 |
| | | 647 | | Scientific | | |
| Strepravidin | | Cy2 | 1:250 | Jackson Immuno | 016-220-084 | 84799 |
| Strepravidin | | Cy3 | 1:300 | Jackson Immuno | 016-160-084 | 125000 |
| Strepravidin | | Daylight 649 | 1:300 | Vector Labs | SA-5649 | Y0718, |
| | | | | | | Z0320 |

| | | | | | |
|---|---|---|---|---|---|
| TSA Biotin Kit | HRP | 1:100 | Perkin Elmer | NEL700 | 2075089 |
| Strepravidin | | | | AD01KT | |
| Tyramide Plus | Cy3 | 1:300 | Perkin Elmer | NEL 7440 | 2310803 |
| | | | | 01KT | |

| | | | |
|---|---|---|---|
| Vectastain Elite ABC Reagent | Vector Labs | PK-7100 | ZD0404 |

### Imaging

Thin sections were imaged on an Olympus BX51 upright microscope and cultured cells were imaged on an Olympus IX71 inverted microscope using Openlab software (PerkinElmer). Chromogenic stains were imaged on a Leica DM 1000 LED. Fiji⁵⁰ and/or Photoshop (Adobe) were used to process (brightness, contrast and gamma) and merge channels. Thick sections for 3D analysis of connectivity and clonal analysis were imaged on a Leica upright AOBS confocal microscope and processed and analyzed using Imaris (Bitplane) or Volocity (PerkinElmer) software. For R26R-Confetti^{+/-} mouse and pSMAD3 analysis, images were acquired using a Nikon A1R GaAsP inverted SP confocal microscope and NIS elements software and processed and analyzed using Imaris software. Sirius-red-stained sections were imaged using a Cytation 5 cell imaging multi-mode reader (BioTek).

### Ink Injection

A catheter was inserted in a retrograde fashion into the common bile duct of postmortem mice and waterproof ink (Higgins) was slowly injected. Left liver lobes were dehydrated in 1:1 methanol:water followed by 100% methanol. Ink was visualized by tissue clearing in 1:2 benzyl alcohol:benzyl benzoate (BABB) solution and imaged on a Leica M205A or Nikon SMZ800 stereoscope.

### Cholangiocyte Isolation

Nonparenchymal liver cells were isolated from >P115 Alb-Cre^{+/-};Rbpj^{f/f};Hnf6^{f/f} and Rbpj^{f/f};Hnf6^{f/f} mice as previously described⁵¹. Cells were resuspended at 1×10⁷ cells/mL in DMEM/2% FBS and blocked with Mouse Fc Block (BD Biosciences) for 30 minutes. Cells were incubated with fluorochrome-conjugated antibodies (Table 2) and DBA-FITC (Vector Laboratories) for 30 minutes, washed with cold DPBS 3 times and resuspended in DMEM/2% FBS. Sytox Red (Thermo Fisher Scientific) was added to label dead cells prior to sorting. Unstained and single-stained cells were used for compensation. Specificity of DBA binding was verified with a GalNAc (Sigma)-blocked control as previously described⁵². Cells were analyzed and sorted on a FACSAria III using FACSDiva software (BD Biosciences). From the CD11b⁻ CD31⁻CD45⁻ population, EPCAM⁺DBA⁻ cells were collected as peripheral cholangiocytes and EPCAM⁺DBA⁺ as hilar cholangiocytes. FlowJo (FlowJo, LLC) was used to analyze data and generate charts. Cells were either sorted into DMEM/2% FBS, pelleted and snap frozen, or sorted directly into extraction buffer for RNA purification.

### Hepatocyte Isolation

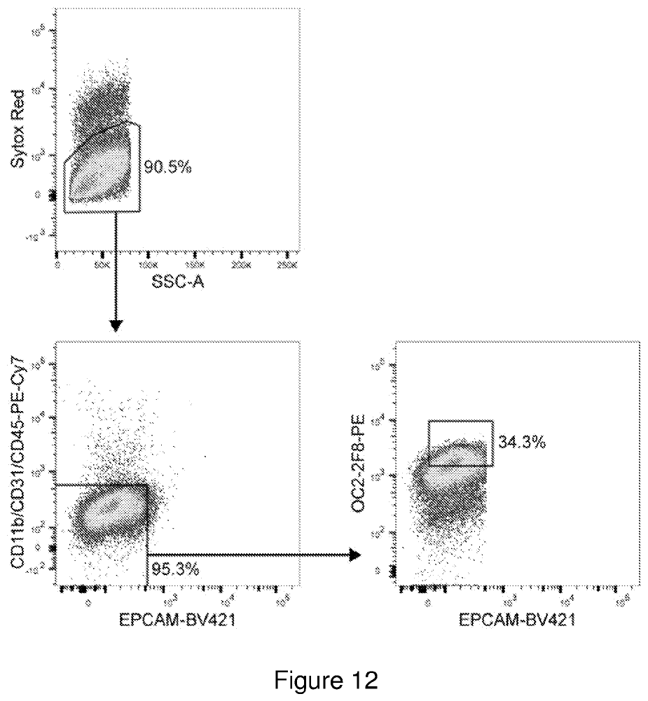

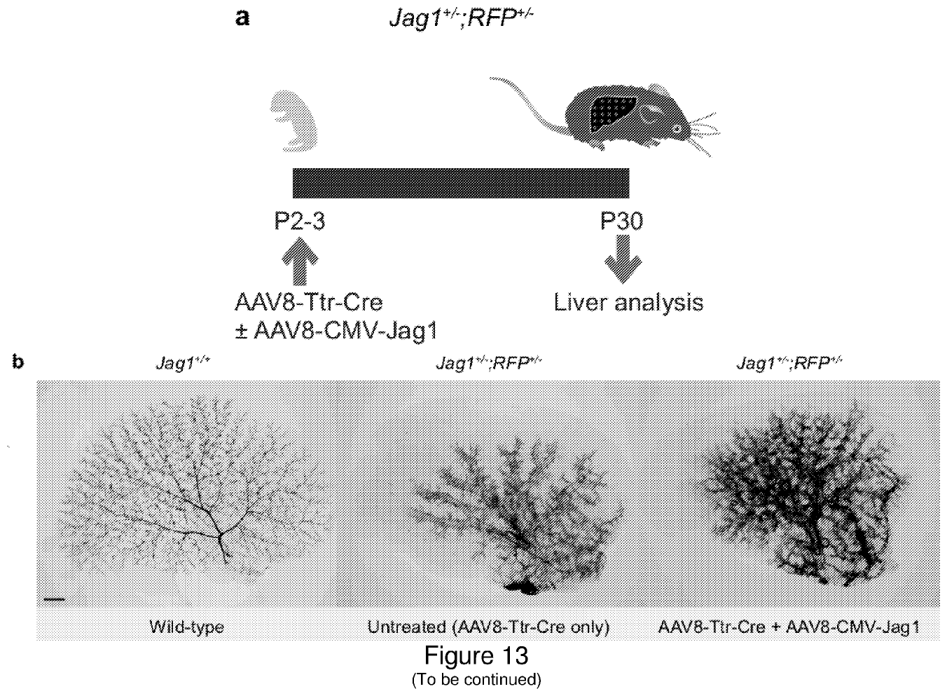

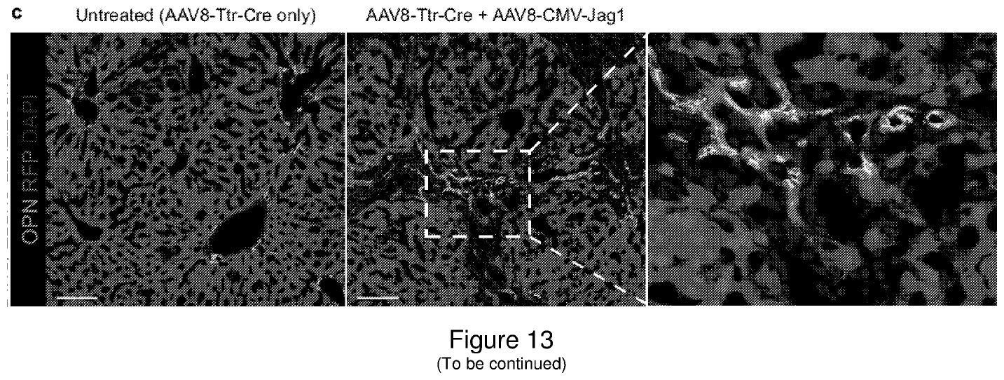

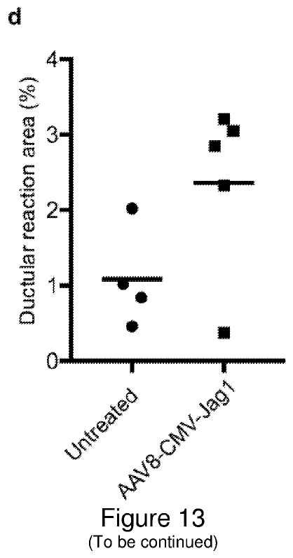

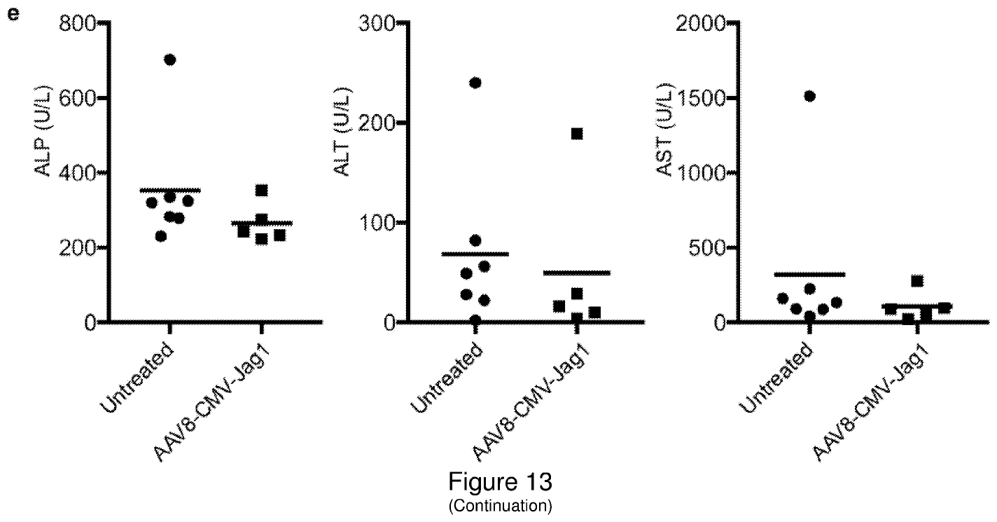

Hepatocytes were isolated from >P115 Alb-Cre^{+/-};Rbpj^{f/f};Hnf6^{f/f} mice by 2-step collagenase perfusion followed by purification through a Percoll gradient. Cells were resuspended at 1×10⁶ cells/100 μL in Hanks Buffer with 10% FBS and incubated with OC2-2F8 antibody for 1 hour on ice. Cells were washed with cold DPBS 2 times and resuspended in Hanks/10% FBS. Fluorochrome-conjugated secondary antibody (Table 3) was added and cells were incubated for 30 minutes on ice followed by 2 washes with cold DPBS. Cells were blocked with 5% normal rat serum (Jackson Immuno) in Hanks Buffer for 10 minutes on ice. Cells were then incubated with fluorochrome-conjugated antibodies (Table 2) for 30 minutes on ice. Cells were then washed in cold DPBS 3 times and resuspended in Williams E medium/2% FBS. Sytox Red (Thermo Fisher Scientific) was added to label dead cells prior to sorting. Unstained and single-stained cells were used for compensation. Cells were analyzed and sorted on a FACSAria III using FACSDiva software (BD Biosciences). From the CD11b⁻CD31⁻CD45⁻EPCAM⁻ population, OC2-2F8⁺ cells were collected as hepatocytes (FIG. 12). Cells were either sorted into DMEM/2% FBS, pelleted and snap frozen, or sorted directly into extraction buffer for RNA purification.

### Hepatocyte Transplantation

Hepatocytes were isolated from donor mice by 2-step collagenase perfusion followed by purification through a Percoll gradient. 1×10⁶ viable cells were resuspended in 80-100 μl of Williams E Medium with glutamine. Transplantation was performed by transdermal intrasplenic injection of the cell suspension under isoflurane anesthesia.

### DDC Diet Feeding

Mice received PicoLab Mouse Diet 20, 5058 (LabDiet) with 0.1% 3,5-diethoxycarbonyl-1,4-dihydrocollidine (DDC; Sigma-Aldrich) for the indicated durations.

qPCR and Gene Expression Analysis

Genomic DNA was isolated from cells using QIAamp DNA Micro Kit or DNeasy Blood & Tissue Kit (Qiagen). RNA was extracted using Trizol Reagent (Thermo Fisher Scientific) and purified by isopropanol precipitation (cells in collagen gel) or purified using RNAeasy Mini Kit (Qiagen) (liver tissue). Reverse transcription was performed using qScript cDNA Supermix (Quanta Biosciences). qPCR was performed using SYBR green reagent (Thermo Fisher Scientific) in a ViiA 7 system (Thermo Fisher Scientific). Reactions were performed in triplicate, and expression was normalized to an Rbpj (genotyping) or Gapdh (Glyceraldehyde-3-phosphate Dehydrogenase; gene expression) reference and quantified using the ΔΔCt method. Primers are listed in Table 4.

TABLE 4
qPCR Primers.

| Target | Forward primer (5′-3′) | Reverse primer (5′-3′) |
|---|---|---|
| Alb | gcagatgacagggcggaacttg | aaaatcagcagcaatggcaggc |
| Epcam | gcggctcagagagactgt | ccaagcatttagacgccagttt |
| Fah | accataggagactacacggact | accacaatggaggaagctcg |
| Gapdh | catggccttccgtgttcct | gcggcacgtcagatcca |
| Hnf4a | catcaacgaccggcagtac | gcagcaggttgtcaatcttg |
| Krt19 | agagacggaggcccgttat | ctggttctggcgctctatgtc |
| Rbpj (floxed) | gagatagaccttggtttgttg | ccactgttgtgaactggcgt |
| Rbpj (reference) | gccactccatgtccaaaaga | gcaagttatagctcagaacagcaa |
| Sox9 | agaccagtacccgcatctgcac | tctcttctcgctctcgttcagca |
| Spp | actgccaatctcatggtcgt | actgccaatctcatggtcgt |
| Ttr | tggacaccaaatcgtactgg | aattctgggggttgctgac |

### RNA Sequencing (RNA-seq)

RNA was purified from FACS-isolated cells using PicoPure Kit (Thermo Fisher Scientific). RNA quality was assessed using RNA 6000 Pico Kit on a 2100 Bioanalyzer (Agilent). Samples with RIN≥7.7 and at least 15 ng of RNA were used to construct sequencing libraries using Clontech Low Input Library Prep Kit v2. Libraries were sequenced on a HiSeq 3000, 10 samples per lane, with single-end 50 bp reads. Raw reads were aligned to the mm10 mouse genome with annotations provided by UCSC using CobWEB, a proprietary Burrows-Wheeler Transform method. Reads per kb per million (RPKM) were calculated from aligned reads using the expectation-maximization algorithm. RPKM was thresholded at 1, log 2 transformed, normalized using the DESeq algorithm and baselined to the median of all samples. Analyses were performed on transcripts with RPKM>5 in all samples of at least 1 experimental condition (n=17,793 transcripts). These reasonably expressed transcripts were used in principle-component analysis. All transcripts with fold change >3 in at least 1 of the 3 possible pairwise comparisons (N=6,464) were selected, and a 1-way ANOVA was performed to identify significantly differential genes with FDR-corrected P<0.05 (n=4,997). Venn diagrams were used to identify unique and shared gene signatures. Gene sets were submitted to ToppGene.cchmc.org for identification of pathway and biological process enrichments.

### Western Blot

Nuclear and cytoplasmic extracts were generated from whole liver using previously described buffers with protease and phosphatase inhibitors⁵³. Samples were run on SDS-PAGE 4-20% Tris-Glycine gradient gels, electrophoretically transferred to nitrocellulose membrane and probed with antibodies. Signals were detected by ECL Western blotting substrate (GE Healthcare). The membrane was stripped and reprobed with anti-actin antibody to verify and normalize protein loading using densitometry. Quantification was performed using ImageJ software.

### Serum Chemistry

Blood was collected from postmortem Alb-Cre^{+/-};Rbpj^{f/f};Hnf6^{f/f} mice and controls of ages P20 to ≥P150 and tested for serum total bilirubin (TecoDiagnostics) and ALP, ALT and AST (ADVIA XPT clinical chemistry system, Siemens). For serial bilirubin measurements, blood was collected by retro-orbital venipuncture and tested for total bilirubin every 2 weeks from P60 to P189 from 4 different litters. Blood was collected from Alb-Cre^{+/-}; Rbpj^{f/f};Hnf6^{f/f};Tgfbr2^{f/f} mice and controls by retro-orbital venipuncture and tested for serum total bilirubin (TecoDiagnostics). Blood was collected from Alb-Cre^{+/-};Rbpj^{f/f};Hnf6^{f/f} mice intravenously injected with AAV8-Ef1α-caTgfbr1 and controls by retro-orbital venipuncture and serum total bilirubin was measured as previously reported⁵⁴ using a Synergy 2 microplate reader (BioTek). Serum absorbance at 540 nm was subtracted from serum absorbance at 450 nm. A linear trendline equation of serum absorbance versus serum total bilirubin was determined by independently measuring absorbance and total bilirubin (TecoDiagnostics) of known cholestatic and noncholestatic serum samples. This equation was used to convert absorbance readings to serum total bilirubin levels.

### In Vitro Conversion Assay

In vitro 3D culture of hepatocytes to induce biliary conversion was carried out as previously reported²¹ with the following modifications. Hepatocytes were isolated from 8-10-week-old Rbpj^{f/f};Hnf6^{f/f} mice injected with 1×10¹² vgs of AAV8-Ttr-Cre 2 weeks prior to delete RBPJ and HNF6. Hepatocytes were isolated by 2-step collagenase perfusion followed by purification through a Percoll gradient, all in the absence of serum. Cells were grown on Primaria plates for 6 days to form spheroids. Spheroids were cultured in collagen gels (Cultrex Rat Collagen I, Lower Viscosity, Trevigen) in the presence of 10 μM SB-431542 (Selleckchem) or vehicle (DMSO). The medium was changed every other day.

### Quantification and Statistics

For sparse-labeling clonal analysis, 8 liver regions from each of 3 Alb-Cre^{+/-}; Rbpj^{f/f};Hnf6^{f/f};R26ZG^{+/+} mice were analyzed. GFP-positive cells in all clones within 100 μm-thick sections were manually counted using Imaris software for visualization. GFP-positive cells in direct contact were considered clones. Clones that extended to and potentially beyond the x, y or z boundaries were excluded. For clonal analysis in R26R-Confetti^{+/-} mice, 30-40 μm z-stack images of wsCK-positive DBA-negative pBDs (peripheral bile ducts) and wsCK-positive DBA-positive hBDs were visualized in 3D with Imaris software. The module Surfaces within Imaris was used to render a 3D surface created on an intensity value on a per channel basis. The rendered surface-per-channel was masked to the Hoechst nuclear stain. 3D pBD masks were used to manually count cells per clone. Multiple portal regions were analyzed for clones at P90 (14 and 17 per Alb-Cre^{+/-};Rbpj^{f/f};Hnf6^{f/f};R26R-Confetti^{+/-} and 8 and 9 per Alb-Cre^{+/-};R26R-Confetti^{+/-} mouse) and P150 (7-10 per Alb-Cre^{+/-};Rbpj^{f/f};Hnf6^{f/f};R26R-Confetti^{+/-} and 8-10 per Alb-Cre^{+/-};R26R-Confetti^{+/-} mouse). To determine labeling efficiencies in Alb-Cre^{+/-}; Rbpj^{f/f};Hnf6^{f/f};R26ZG^{+/+} mice, for each mouse, 20 random fields were analyzed for wsCK-positive DBA-negative peripheral cholangiocytes (about 350 cells) and 5 random fields were analyzed for hepatocytes (about 1,500 cells).

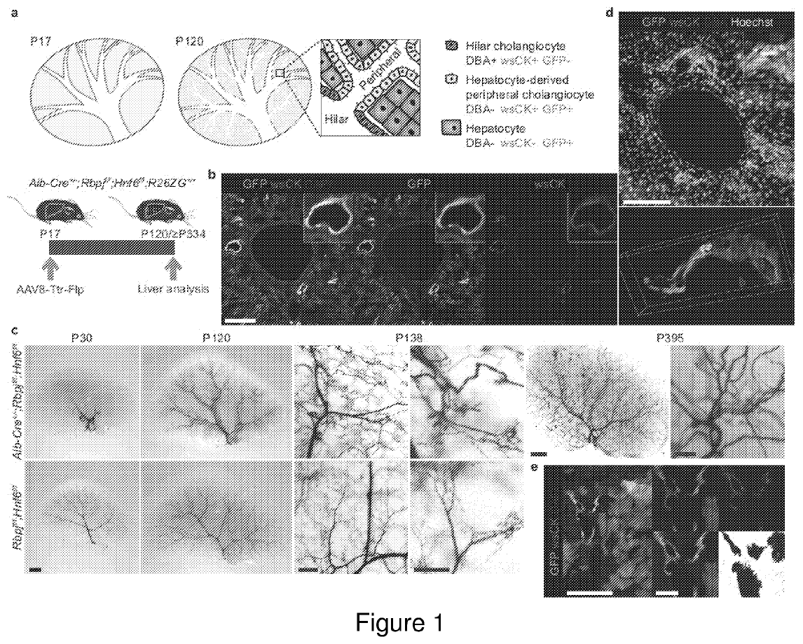

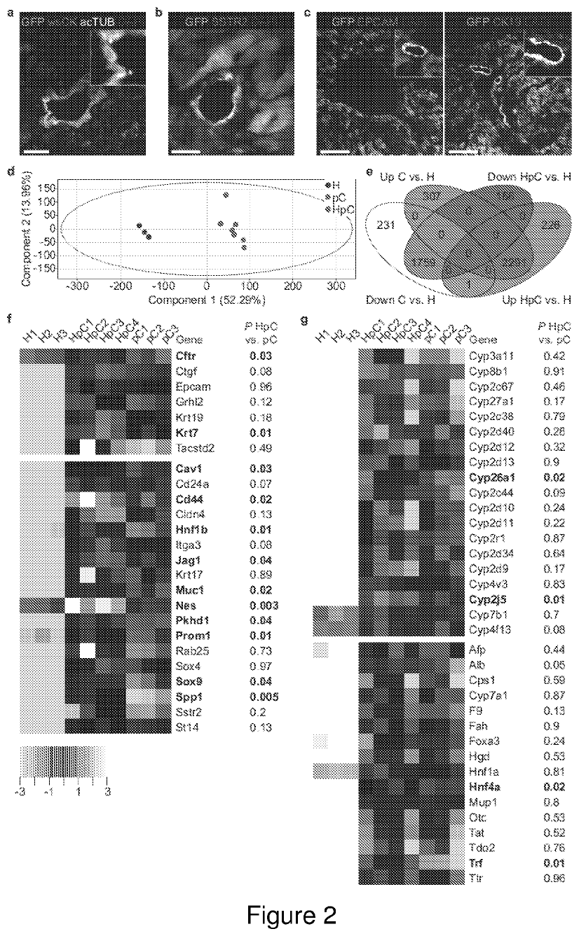

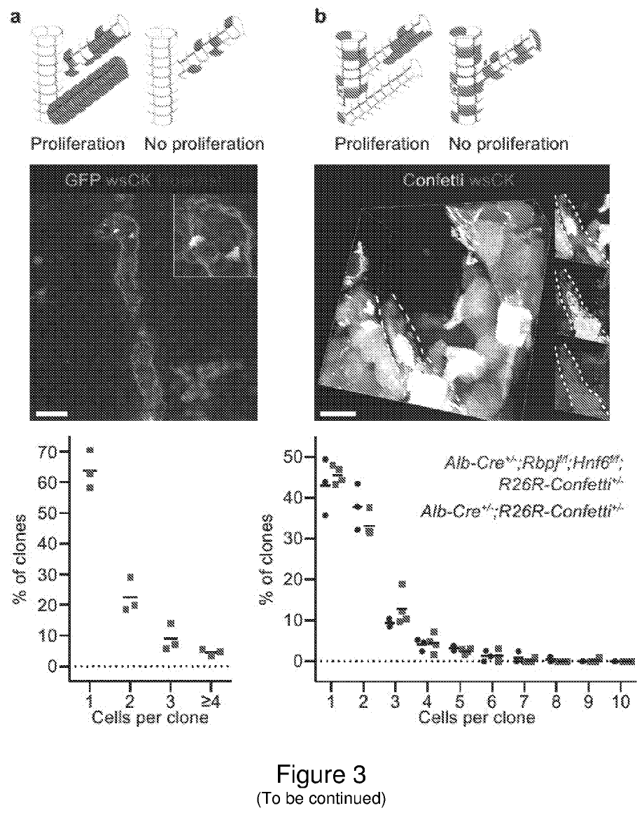

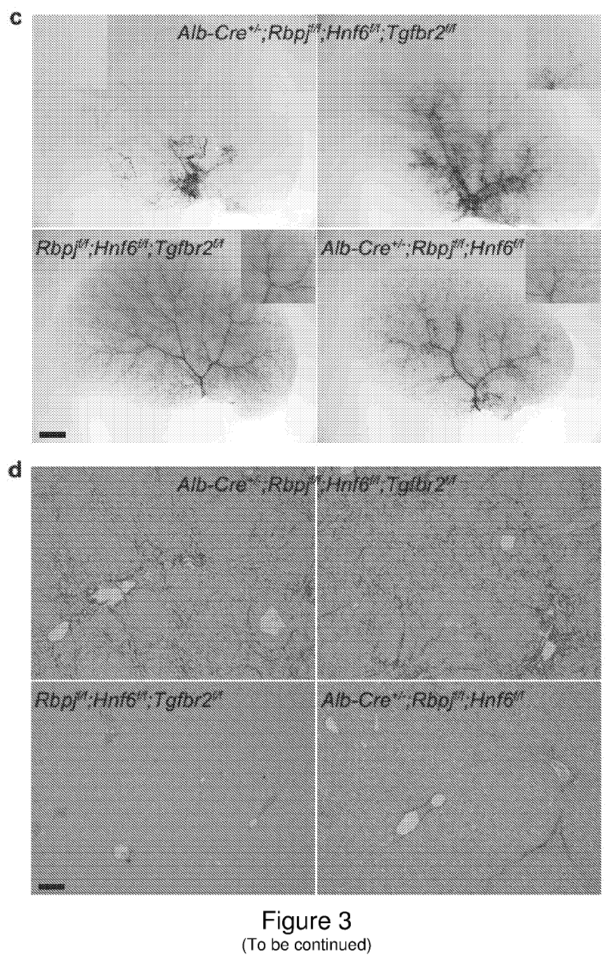

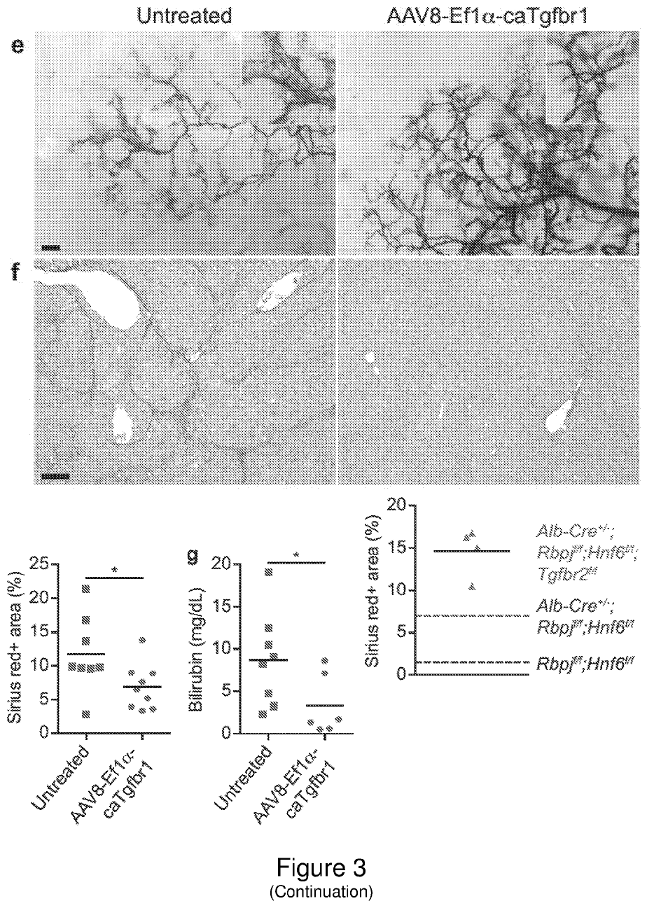

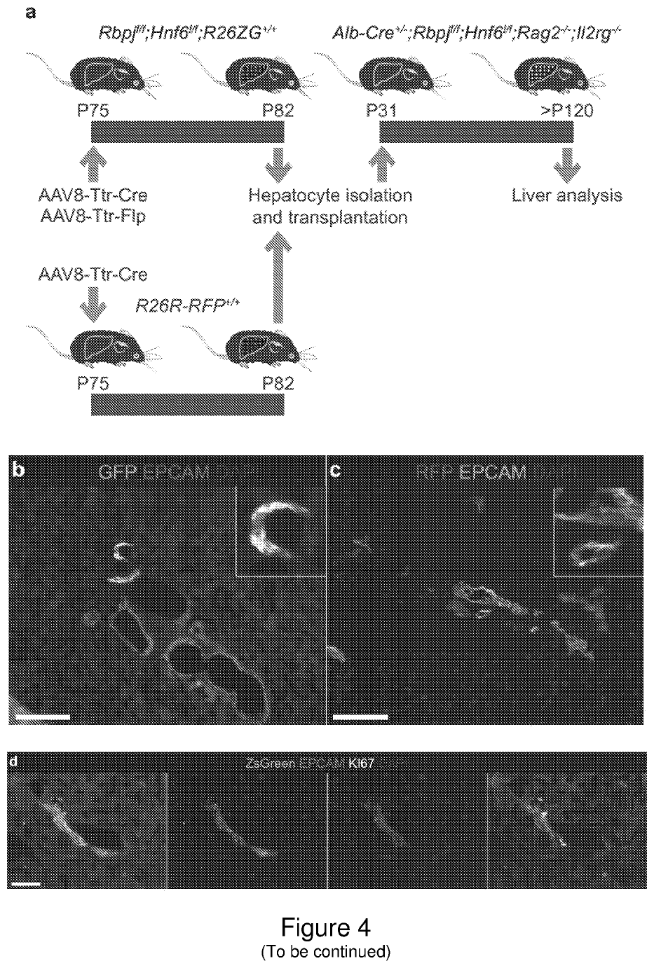

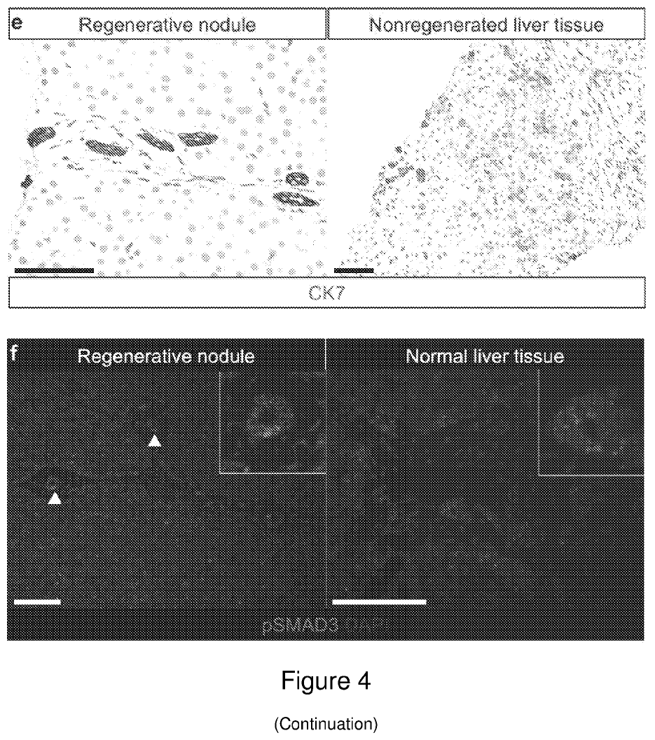

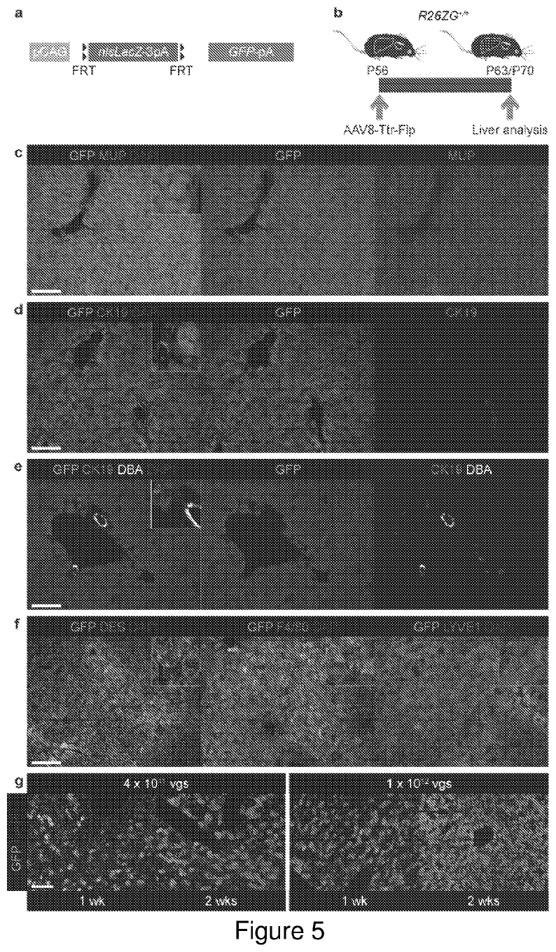

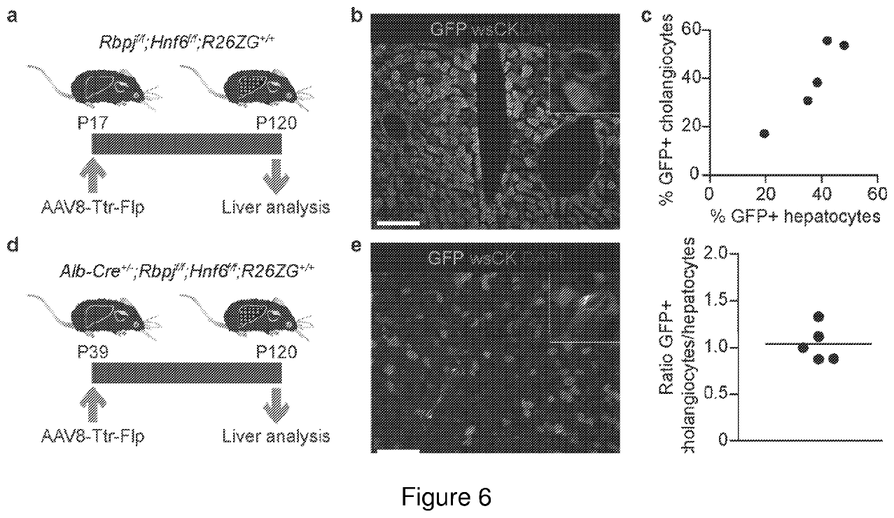

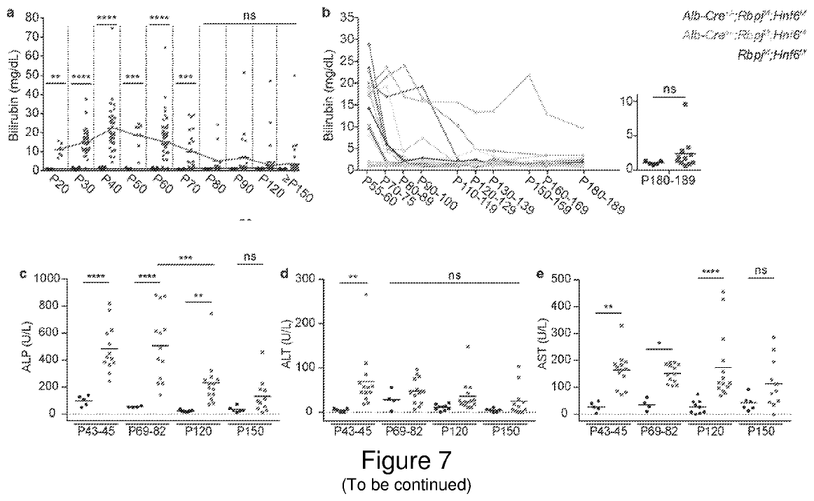

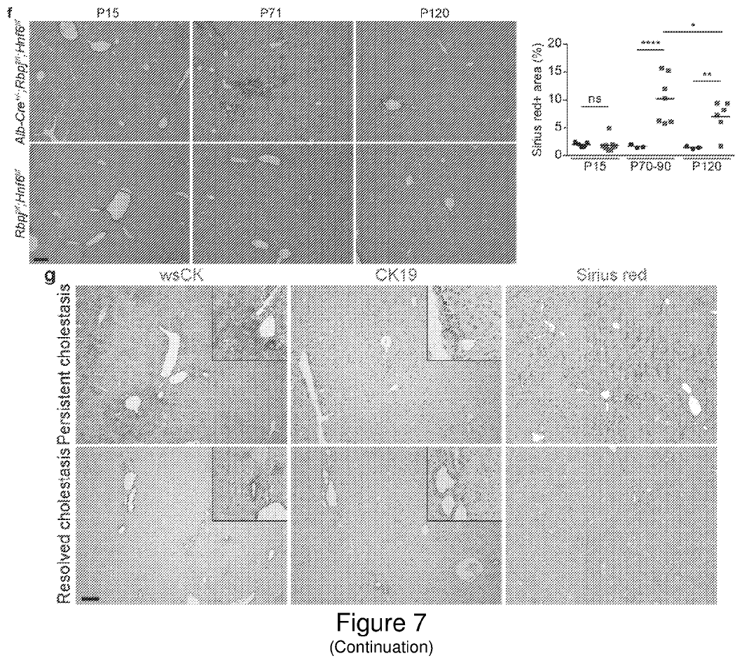

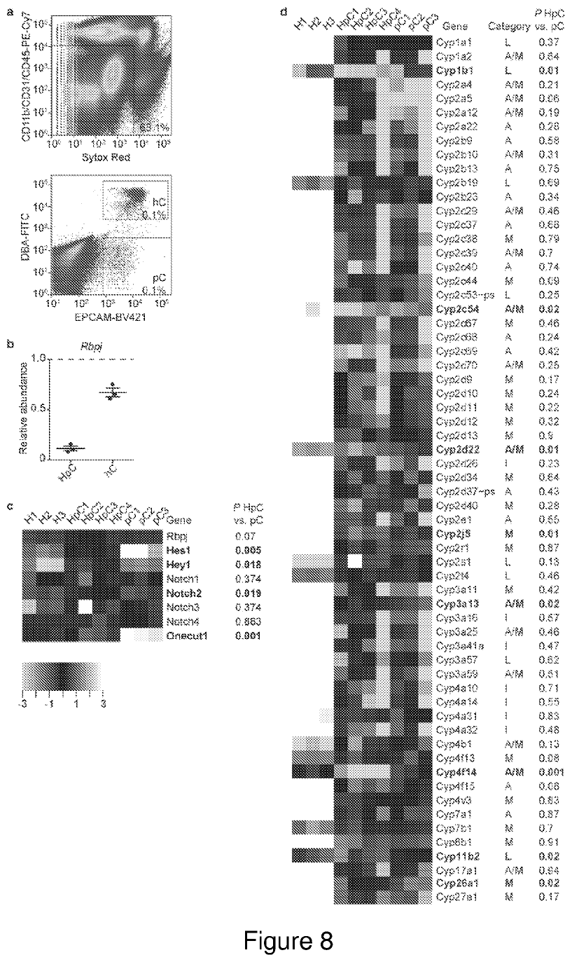

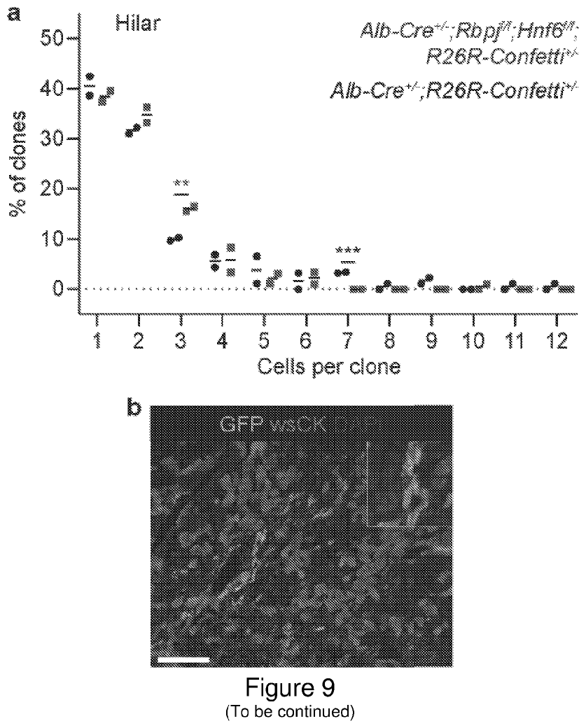

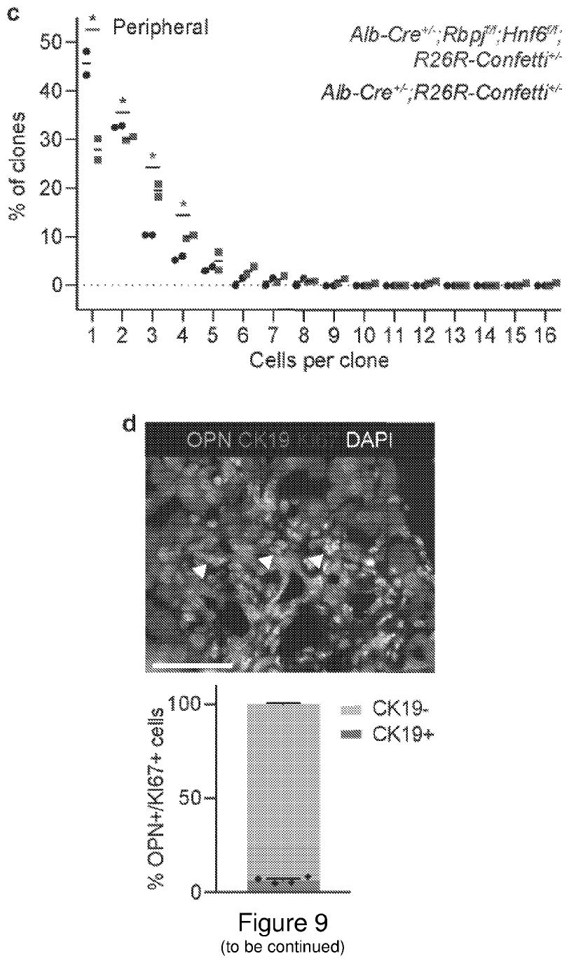

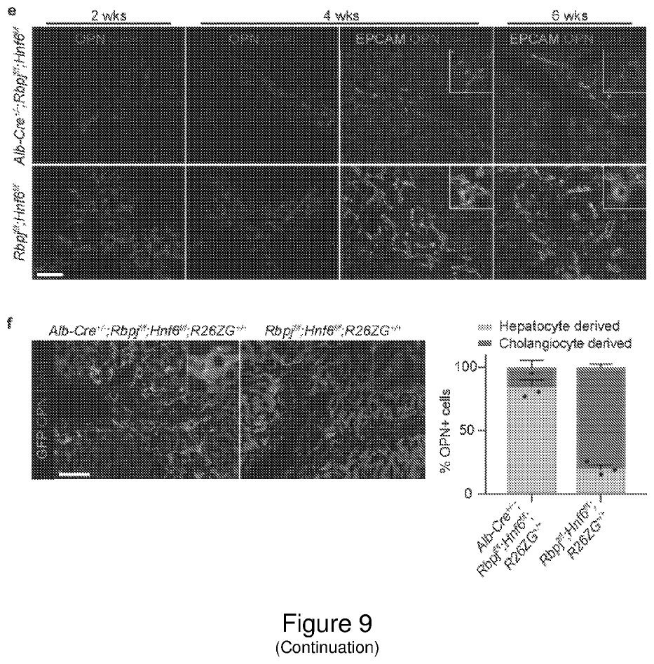

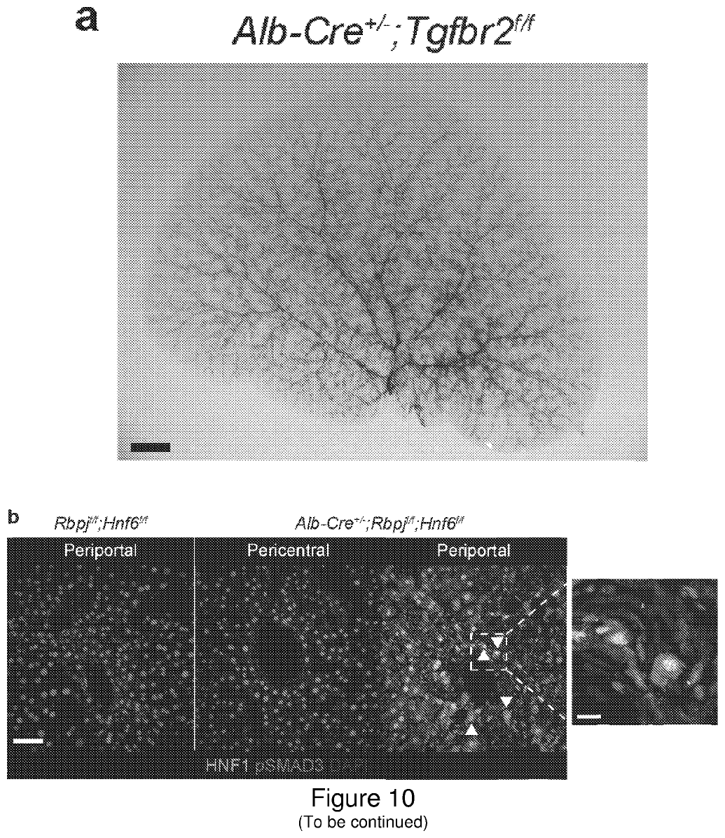

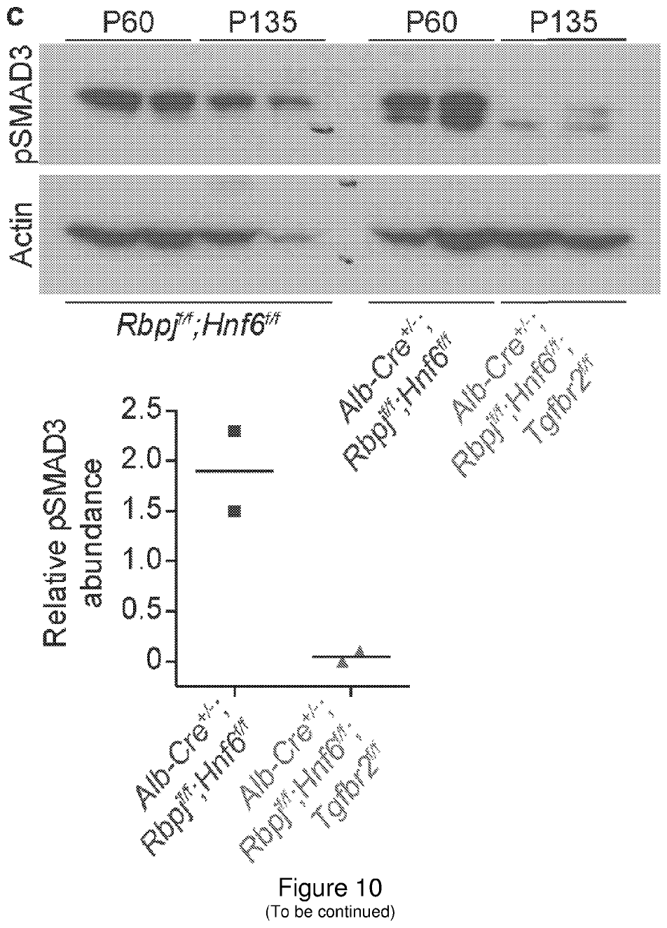

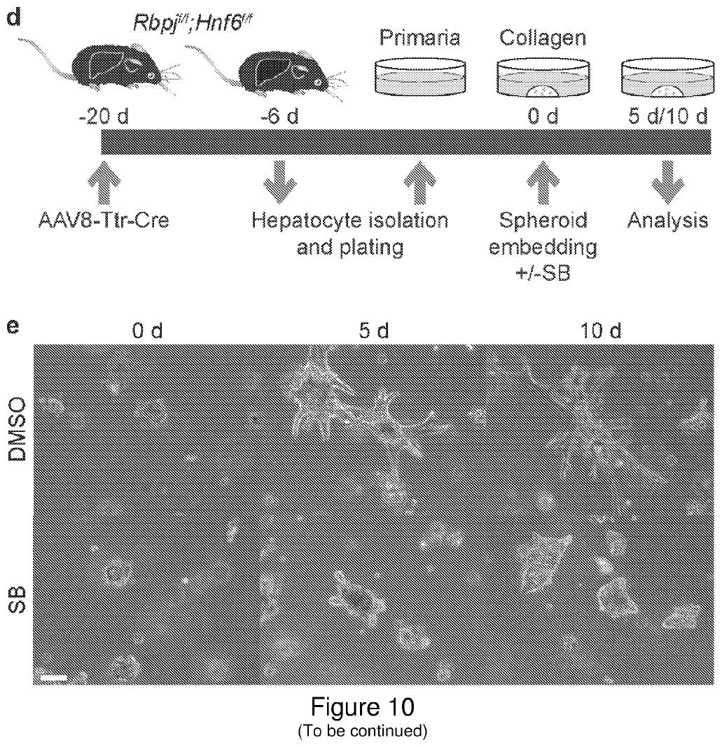

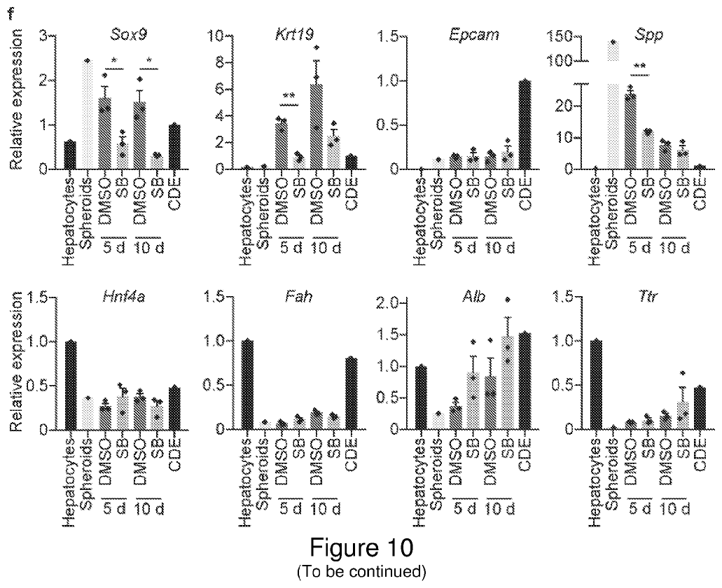

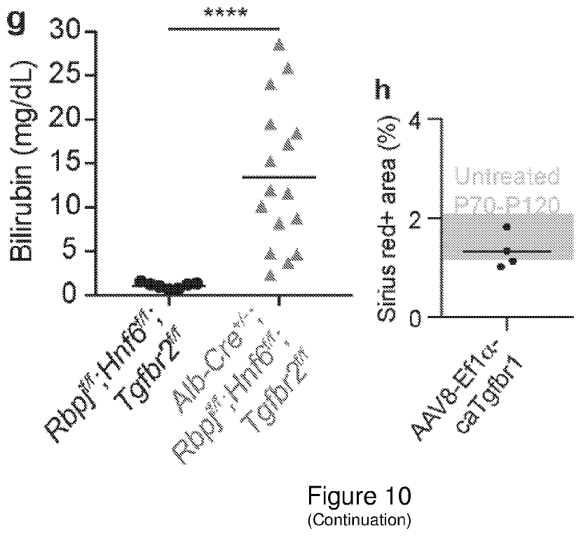

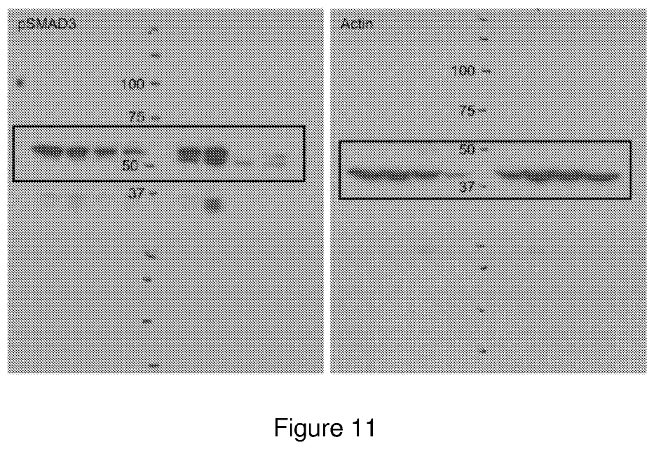

For proliferation analysis, OPN-positive cells in 4 random portal fields from each of 4 mice were analyzed for KI67 and CK19 staining. For hepatocyte-fate-tracing analysis in DDC diet-fed mice, 10 random portal fields from 4 Alb-Cre^{+/-};Rbpj^{f/f};Hnf6^{f/f};R26ZG^{+/+} and 3 Rbpj^{f/f};Hnf6^{f/f};R26ZG^{+/+} mice were analyzed. For quantification in transplantation experiments, all portal areas with donor-derived cells in a section from at least 3 lobes per mouse were examined and scored for the presence or absence of donor hepatocyte-derived EPCAM-positive cholangiocytes. For human samples, 63 bile ducts between the 2 patient samples were scored for the presence of nuclear pSMAD3. For Sirius-red-staining quantification, liver samples were stained in batches and sections of whole lobes were imaged at equal exposure. Using Fiji software, a threshold was set for Sirius-red-positive area within each lobe and the percent of the total area that was Sirius red positive was measured and is reported. Charts were generated in Prism 6 or 7 (GraphPad). Researchers were not blinded when analyzing results. P<0.05 was considered statistically significant. Experiments were replicated independently once (FIG. 2a, b, d-g; FIG. 3a, b, d, g; FIG. 5c-g; FIG. 2c, e; FIG. 7a-g; FIG. 8b-d; FIG. 9a, c, d, f; FIG. 10c, g; Table 1), at least twice (FIG. 1d, e; FIG. 2c; FIG. 3f; FIG. 4b-f; FIG. 9b, e; FIG. 10a, b, e, f, h) or at least thrice (FIG. 1b, c; FIG. 3c, e; FIG. 6b; FIG. 8a).

TABLE 1
Characterization of human subjects and samples.

| Human | | ALGS | ALGS | Age at liver | Liver |
|---|---|---|---|---|---|
| subject | Gender | genotype | phenotype | analysis | samples |
| ALGS | Male | Heterozygous JAG1 | Neonatal cholestasis, bile | 3 years | Regenerative nodule and |
| patient | | exon 1-26 deletion | duct paucity, liver fibrosis, | | nonregenerated tissue |
| | | | portal hypertension, | | (segment [symbol] of right lobe and |
| | | | hepatocellular carcinoma, | | segment 8 of right lobe of |
| | | | liver transplantation, left | | expla [symbol] liver, respectively) |
| | | | pulmonary artery stenosis, | | |
| | | | butterfly vertebrae, facial | | |
| | | | features | | |
| ALGS | Male | Heterozygous JAG1 | Failure to [symbol] | 15 years | Regenerative nodule and |
| patient | | exon 4 mutation | hepatomegaly, bile duct | | nonregenerated tissue |
| | | ( [symbol] p.W167R) | paucity, cholestasis, liver | | (biopress of segment 3 of left |
| | | | fibrosis; left pulmonary artery | | liver lobe and segment 5 of |
| | | | stenosis; facial features | | right liver lobe, respsectively) |
| Normal | Male | Normal JAG1 and | None | 35 years | [symbol] normal tissue |
| control | | NOTCH2 sequences | | | (resection of segment 4 of left |
| | | | | | liver lobe for metastasis of |
| | | | | | rectaladenocarcinoma) |

[symbol]

indicates data missing or illegible when filed

### Data Availability

The Gene Expression Omnibus accession number for the RNA-seq data is GSE108315.

### Example 2

All published studies of hepatocyte plasticity used animals with a fully developed biliary system in which residual cholangiocytes were available to regenerate injured bile ducts, likely leading to insufficient pressure for transdifferentiation. To determine the full extent of hepatocyte plasticity, we used mice lacking the intrahepatic biliary system.

Specifically, we used mice that mimic the hepatic phenotype of ALGSa human disease caused by impaired NOTCH signaling²²⁻²⁴—generated by deletion of floxed alleles of the NOTCH signaling effector RBPJ and, to prevent compensation²⁵, the transcription factor HNF6 in embryonic liver progenitors using Cre expressed from an albumin promoter (Alb)². These Alb-Cre^{+/-};Rbpj^{f/f};Hnf6^{f/f} mice are severely cholestatic because they lack peripheral bile ducts (pBDs), the branches of the intrahepatic biliary tree, at birth. Greater than 90% of the mice survive, however, and form pBDs and a functional biliary tree by postnatal day (P) 120²⁶ (FIG. 1a). Although Alb-Cre^{+/-};Rbpj^{f/f};Hnf6^{f/f} mice have hilar bile ducts (hBDs), the trunk of the biliary tree, it was expected that the new pBDs would originate from hepatocytes because the livers of Alb-Cre^{+/-};Rbpj^{f/f};Hnf6^{f/f} mice contain hybrid cells expressing hepatocyte and biliary markers^{2,26}. To confirm this expectation, we developed Cre-independent hepatocyte fate tracing. For this, we activated the Flp-reporter GFP gene in R26ZG^{+/+} mice specifically in hepatocytes by intravenous injection of an adeno-associated virus (AAV) serotype 8 vector expressing Flp from the transthyretin promoter (FIG. 1a and FIG. 5a-g).

### Example 3

To trace hepatocytes during pBD formation, we intravenously injected Alb-Cre^{+/-}; Rbpj^{f/f};Hnf6^{f/f};R26ZG^{+/+} mice with AAV8-Ttr-Flp at P17, i.e., before cells expressing biliary markers emerge in the liver periphery^{2,26}. At P120, the newly formed pBDs—identified by wsCK IF—were GFP positive, indicating hepatocyte origin (FIG. 1a, b). In contrast, pBDs that formed normally during liver development in Cre-negative littermates were GFP negative (FIG. 6a, b). In many pBDs of Alb-Cre^{+/-};Rbpj^{f/f};Hnf6^{f/f};R26ZG^{+/+} mice all cells were GFP positive, while the overall labeling efficiency of peripheral cholangiocytes was 39.2±7.2%; however, this number correlated well with the hepatocyte labeling efficiency (36.6±4.8%; paired 2-sided Student's t-test; P=0.48) (FIG. 6c), indicating that all peripheral cholangiocytes originated from hepatocytes. We also observed hepatocyte-derived peripheral cholangiocytes in mice in which hepatocytes were labeled after weaning (P39) (FIG. 6d, e). These results show that hepatocytes can form pBDs de novo.

### Example 4

To investigate the function of the hepatocyte-derived pBDs (HpBDs), we determined if they are contiguous with the extrahepatic biliary system. At P30, retrograde ink injection into the common bile duct filled only hBDs, reflecting a severely truncated biliary tree lacking pBDs; however, ink injection at ≥P120 revealed a biliary tree of normal dimensions, demonstrating that HpBDs are connected to and patent with hBDs (FIG. 1c). 3D reconstruction of confocal imaging confirmed that HpBDs form contiguous lumens with DBA-labeled hBDs (FIG. 1d). Accordingly, HpBDs were effective in draining bile, as evidenced by normalization of serum markers of cholestasis (total bilirubin and ALP; FIG. 7a-c) and hepatocyte injury (ALT and AST; FIG. 7d, e) and resolution of cholestasis-induced liver fibrosis (Sirius-red staining; FIG. 70. A few mice that continued to have elevated serum total bilirubin and liver fibrosis showed abundant wsCK-positive hepatocytes but no HpBDs (FIG. 7a, b, g). In contrast to the transient hepatocyte-derived ductules observed in other mouse models of cholestatic liver injury³⁻⁶, HpBDs were stable beyond the time when cholestasis and liver injury were resolved and were maintained for life (≥P334) (FIG. 1c, e). These results show that HpBDs provide normal and stable biliary function.

### Example 5

We also investigated the authenticity and maturity of the cholangiocytes forming the HpBDs. The cells displayed acetylated tubulin (acTUB)-marked primary cilia (FIG. 2a), indicating a switch from hepatocyte to biliary fate¹⁹, and somatostatin receptor 2 (SSTR2) (FIG. 2b), a marker of biliary function²⁷. They also expressed the mature biliary markers EPCAM and CK19 (FIG. 2c), which are lacking or expressed at low levels in hepatocyte-derived metaplastic biliary cells^{3,5}. To substantiate these results, we performed RNA sequencing (RNA-seq) on hepatocyte-derived peripheral cholangiocytes isolated as EPCAM-positive DBA-negative cells by FACS from >P115 Alb-Cre^{+/-};Rbpj^{f/f};Hnf6^{f/f} mice (FIG. 8a). RBPJ and HNF6 were deleted in these cells (FIG. 8b, c), which was expected because Alb-Cre is active in embryonic liver progenitors before they commit to hepatocyte or biliary fate^{2,9}. Principal-component analysis showed that hepatocyte-derived peripheral cholangiocytes cluster closely with normal peripheral cholangiocytes isolated from Cre-negative littermates, but not with parental hepatocytes (FIG. 2d, e and Supplementary Table 1). Accordingly, hepatocyte-derived peripheral cholangiocytes expressed normal levels of mature biliary markers that are virtually undetectable in 3,5-diethoxycarbonyl-1,4-dihydrocollidine (DDC) diet-induced hepatocyte-derived metaplastic biliary cells³, except for CFTR, which is less enriched in cholangiocytes than the other markers (FIG. 20. Hepatocyte-derived peripheral cholangiocytes also expressed other commonly used markers of biliary differentiation^{3,5,20,25,27,28} at normal or near-normal levels (FIG. 2f), and showed virtually no memory of hepatocyte differentiation^{3,29,30} (FIG. 2g and FIG. 4d). These results show that hepatocyte-derived peripheral cholangiocytes are authentic and mature peripheral cholangiocytes.

### Example 6

The contribution of proliferation to HpBD formation, i.e., whether a few hepatocytes proliferate extensively after transdifferentiation, or whether many hepatocytes transdifferentiate, was also investigated. For this, we measured clonal expansion in HpBDs by sparsely labeling hepatocytes in Alb-Cre^{+/-};Rbpj^{f/f};Hnf6^{f/f};R26ZG^{+/+} mice with low-dose AAV8-Ttr-Flp at P18 and quantifying the cells in GFP-positive clones at P120 (FIG. 3a). Surprisingly, we found only 1.56±0.05 cells/clone in HpBDs, with 1-cell clones accounting for 63.8±3.6% of clones. To exclude that the nonintegrating AAV vector missed proliferating hepatocytes, we analyzed clone size in Alb-Cre^{+/-};Rbpj^{f/f};Hnf6^{f/f};R26R-Confetti^{+/-} mice in which hepatocytes and hilar cholangiocytes are stochastically labeled with 1 of 4 fluorescent proteins (FIG. 3b). We found 1.91±0.06 cells/clone and 45.6±1.1% 1-cell clones in HpBDs at P150. In addition, the number of cells per clone in hBDs was similar between these mice and Alb-Cre^{+/-};R26R-Confetti^{+/-} control mice at P90 (2.08±0.08 and 2.41±0.01; 2-sided Student's t-test; P=0.060) (FIG. 9a). These results show that HpBDs are polyclonal, i.e., form with little proliferation, and confirm that hilar cholangiocytes do not contribute to HpBD formation.

Unlike in HpBDs, we found significant proliferation in hepatocyte-derived reactive ductules, which are detectable in Alb-Cre^{+/-};Rbpj^{f/f};Hnf6^{f/f};R26ZG^{+/+} mice during cholestasis (FIG. 9b). Clonal analysis showed 2.70±0.08 cells/clone in reactive ductules in P90 Alb-Cre^{+/-}; Rbpj^{f/f};Hnf6^{f/f};R26R-Confetti^{+/-} mice, with more 3- and 4-cell clones than in pBDs in Alb-Cre^{+/-}; R26R-Confetti^{+/-} mice (FIG. 9c). Because HpBDs form with little proliferation, we reasoned that these proliferating cells are hepatocyte-derived metaplastic biliary cells³⁻⁶. Indeed, we found that 93.51±0.81% of the proliferating biliary cells identified by KI67 and osteopontin (OPN) IF in Alb-Cre^{+/-};Rbpj^{f/f};Hnf6^{f/f} mice near the peak of cholestasis (P54) were CK19 negative (FIG. 9d).

### Example 7

Proliferation in established HpBDs by inducing cholestatic liver injury with DDC diet in >P120 Alb-Cre^{+/-};Rbpj^{f/f};Hnf6^{f/f} mice was also assessed experimentally. Reactive ductules formed 2 weeks later than in Cre-negative littermates and consisted mainly of OPN-positive EPCAM-negative cells, indicative of hepatocyte metaplasia (FIG. 9e). Indeed, after feeding DDC diet to Alb-Cre^{+/-};Rbpj^{f/f};Hnf6^{f/f};R26ZG^{+/+} mice in which hepatocyte fate tracing was induced at >P120, we found that 84.5±5.6% of the cells in reactive ductules originated from hepatocytes, in contrast to 20.1±3.0% in Cre-negative littermates (FIG. 9f). These results confirm that NOTCH signaling is important for cholangiocyte proliferation³¹, which explains our finding of a limited role of proliferation in HpBD formation and underscores the authenticity of hepatocyte-derived peripheral cholangiocytes.

As illustrated by lack of pBDs in Alb-Cre^{+/-};Rbpj^{f/f};Hnf6^{f/f} mice (FIG. 1c), NOTCH signaling is needed for bile duct development⁷⁻⁹, raising the question of which signaling pathway drives HpBD formation in its absence (FIG. 8b, c). We focused on TGFβ signaling because it promotes biliary differentiation and morphogenesis in development³², although it is not essential, as shown by development of a normal biliary tree in mice lacking the TGFβ type II receptor (TGFBR2) in embryonic liver progenitors (FIG. 10a). We reasoned that TGFβ signaling is induced in hepatocytes of Alb-Cre^{+/-};Rbpj^{f/f};Hnf6^{f/f} mice because of the paradoxical role of HNF6 in liver development—it activates the biliary transcription factors HNF1β and SOX9^{25,33}, but inhibits TGFBR2^{32,34}. Indeed, gene ontology (GO) term enrichment analysis of our RNA-seq data suggested active TGFβ signaling in hepatocyte-derived peripheral cholangiocytes, but not in normal peripheral cholangiocytes (Supplementary Table 1, Down pC versus HpC). Moreover, we found high levels of phosphorylated SMAD3 (pSMAD3) in nuclei of periportal HNF1-positive epithelial liver cells and in whole-liver nuclear extracts in Alb-Cre^{+/-};Rbpj^{f/f};Hnf6^{f/f} mice at P60 (FIG. 10b, c).

We functionally validated these findings by showing that the TGFβ inhibitor SB-431542 blocks biliary differentiation and morphogenesis of hepatocytes lacking RBPJ and HNF6 in vitro (FIG. 10d-f). Moreover, at >P120, HpBDs were still absent or severely truncated, and liver fibrosis persisted, in 14/16 Alb-Cre^{+/-};Rbpj^{f/f};Hnf6^{f/f};Tgfbr2^{f/f} mice that, like Alb-Cre^{+/-}; Rbpj^{f/f};Hnf6^{f/f} mice, were cholestatic at P34-53 (FIG. 3c, d and FIG. 10c, g).

### Example 8

The findings disclosed in the preceding Examples led us to investigate whether activating TGFβ signaling in hepatocytes enhances HpBD formation. We intravenously injected P19-24 Alb-Cre^{+/-};Rbpj^{f/f};Hnf6^{f/f} mice with an AAV8 vector expressing constitutively active (ca) TGFBR1 from the elongation factor 1α (Ef1α) promoter. At P80-100, 9/11 treated mice had a mature hierarchical biliary network, whereas 9/9 untreated mice still had an immature homogeneous biliary network³⁵ (FIG. 3e). Reflecting improved bile drainage, cholestasis and liver fibrosis resolved faster in treated mice (FIG. 3f, g). We excluded that AAV8-Ef1α-caTgfbr1 is fibrogenic in Rbpj^{f/f};Hnf6^{f/f} mice (FIG. 10h). These results identify TGFβ signaling as the driver of transdifferentiation and morphogenesis in HpBD formation.

To exclude that transdifferentiation is limited to immature hepatocytes, we investigated whether adult hepatocytes can form HpBDs. To bypass potential adaptive processes in development, we deleted RBPJ and HNF6 and activated GFP in hepatocytes of P75 Rbpj^{f/f};Hnf6^{f/f};R26ZG^{+/+} mice by co-injecting AAV8-Ttr-Cre and AAV8-Ttr-Flp and transplanted the cells 1 week later into P31 Alb-Cre^{+/-};Rbpj^{f/f};Hnf6^{f/f};Rag2^{-/-};Il2rg^{-/-} mice (FIG. 4a). After P120, we found donor hepatocyte-derived mature peripheral cholangiocytes in 5.2±0.8% of the portal areas containing donor cells (FIG. 4b). We also transplanted NOTCH signaling-competent wild-type hepatocytes, which produced donor-derived HpBDs in 78.3±13.9% of such portal areas because 31.6±7.9% of the transplanted cells proliferated after transdifferentiation into cholangiocytes (FIG. 4a, c, d). These results show that adult hepatocytes, and transplanted hepatocytes, respond to the transdifferentiation-inducing and morphogenetic signals in the bile-duct-deficient liver and form HpBDs.

To determine whether our findings are relevant for human ALGS, we obtained liver samples from 2 patients who developed regenerative nodules containing pBDs³⁶ (Extended Data Table 1). The regenerative nodules contained CK7-positive pBDs, whereas nonregenerated liver tissue from the same patients showed abundant CK7-positive cells with hepatocyte morphology, indicative of metaplasia¹⁴⁻¹⁷ (FIG. 4e). To determine if TGFβ signaling is active in the new pBDs, we used pSMAD3 IF. We found nuclear localization of pSMAD3 in 56.1±6.1% of the pBDs in regenerative nodules, but not in pBDs in normal human liver (FIG. 4f). These results suggest that the TGFβ-mediated mechanism of HpBD formation identified in Alb-Cre^{+/-}; Rbpj^{f/f};Hnf6^{f/f} mice is also active in some patients with ALGS.

In conclusion, by showing that hepatocytes can convert into mature cholangiocytes and form a functional and stable biliary system, our findings establish that hepatocyte plasticity extends beyond metaplasia to transdifferentiation and provide the first example of mammalian transdifferentiation building an organ structure de novo. Although hilar cholangiocytes are present in our mice, they fail to expand, resulting in severe cholestatic liver injury and strong pressure for hepatocytes to transdifferentiate into peripheral cholangiocytes. Analogously, cholangiocytes transdifferentiate into hepatocytes only if hepatocyte proliferation is completely suppressed³⁷. The failure of hilar cholangiocytes to proliferate is likely caused by high TGFβ signaling in the cholestatic liver³⁸. Accordingly, we identified TGFβ signaling as the driver of hepatocyte transdifferentiation and HpBD formation in our mice and potentially also in patients with ALGS. Unlike for bile duct development⁷⁻⁹, NOTCH signaling is not needed. Using clinically established AAV vectors and hepatocyte transplantation we show that our findings are potentially translatable into therapies for ALGS and other diseases associated with lack of bile ducts.

### Example 9

We also established that expressing JAG1 in hepatocytes induces de novo bile duct formation. For this, we used Jag1^{+/-} mice on the C57BL/6 background that accurately model the genetics of human ALGS. We bred these mice to R26R-RFP^{+/+} mice—also on the C57BL/6 background—to be able to genetically label hepatocytes by Cre recombinase expression. We found that intravenously injecting these Jag1^{+/-};R26R-RFP^{+/-} mice with AAV8-CMV-Jag1 caused hepatocytes—labeled by additional intravenous injection of AAV8-Ttr-Cre—to convert into cholangiocytes and assemble into bile ducts with a much higher frequency than occurred spontaneously in Jag1^{+/-};R26R-RFP^{+/-} mice intravenously injected only with AAV8-Ttr-Cre. The hepatocyte-derived bile ducts in Jag1^{+/-};R26R-RFP^{+/-} mice intravenously injected with AAV8-CMV-Jag1 and AAV8-Ttr-Cre were connected to the pre-existing bile ducts as evidenced by retrograde ink injection into the common bile duct. Moreover, ink injection showed that the biliary system was much denser and extended further into the periphery of the liver lobules than in Jag1^{+/-};R26R-RFP^{+/-} mice intravenously injected only with AAV8-Ttr-Cre. The function of the hepatocyte-derived bile ducts was further illustrated by lower serum levels of cholestasis and liver injury markers in Jag1^{+/-};R26R-RFP^{+/-} mice intravenously injected with AAV8-CMV-Jag1 and AAV8-Ttr-Cre than in Jag1^{+/-};R26R-RFP^{+/-} mice intravenously injected only with AAV8-Ttr-Cre. These results showed that JAG1 does not have to be provided by mesenchymal cells for bile duct formation to occur, as had been thought to be necessary for bile duct development. In contrast, our results establish that hepatocytes can serve as JAG1 ligand providers to adjacent hepatocytes, and that the resulting activation of Notch signaling leads to bile duct formation from these cells. Collectively, these results establish that expressing JAG1 in hepatocytes can be used to overcome bile duct paucity in mice modeling ALGS, thereby providing a curative strategy for patients suffering from this disease.

In addition to JAG1, we contemplate expression of any one or more of the other Notch receptor ligands JAG2, DLL1, DLL3 and DLL4 to induce Notch signaling in hepatocytes in vivo because they compensate for each other. Moreover, we expect that any of Notch receptors NOTCH1, NOTCH2, NOTCH3, NOTCH4 could be expressed to induce Notch signaling in hepatocytes in vivo, provided surrounding cells express JAG2, DLL1, DLL3 or DLL4. Alternatively, the respective NOTCH intracellular domains, which do not require ligand-mediated activation, could be expressed to induce Notch signaling in hepatocytes in vivo.

### Example 10

As an alternative to AAV vectors and other viral vectors, it is contemplated that nonviral methods, including mRNAs, small activating RNAs or recombinant proteins, could be used to express JAG1, JAG2, DLL1, DLL3, DLL4, NOTCH1, NOTCH2, NOTCH3, NOTCH4 or the respective NOTCH intracellular domains in hepatocytes in vivo. In addition, small molecules could be used to induce Notch signaling or TGFβ signaling in hepatocytes in vivo. These nonviral methods proved to be safe and effective in clinical trials for therapy of genetically encoded liver diseases or hepatocellular carcinoma.

### Example 11

In ALGS patients, CRISPR or CRISPR-mediated activation (CRISPRa) could be used to correct the mutated JAG1 allele or induce the expression of the normal JAG1 allele, respectively. The components of CRISPR or CRISPRa could be delivered to hepatocytes in vivo using viral vectors, particularly AAV vectors, which proved to be safe and effective in clinical trials for therapy of genetically encoded liver diseases.

### REFERENCES

1 Merrell, A. J. & Stanger, B. Z. Adult cell plasticity in vivo: de-differentiation and transdifferentiation are back in style. Nat Rev Mol Cell Biol 17, 413-425 (2016).
2 Vanderpool, C. et al. Genetic interactions between hepatocyte nuclear factor-6 and Notch signaling regulate mouse intrahepatic bile duct development in vivo. Hepatology 55, 233-243 (2012).
3 Tarlow, B. D. et al. Bipotential adult liver progenitors are derived from chronically injured mature hepatocytes. Cell Stem Cell 15, 605-618 (2014).
4 Sekiya, S. & Suzuki, A. Hepatocytes, rather than cholangiocytes, can be the major source of primitive ductules in the chronically injured mouse liver. Am J Pathol 184, 1468-1478 (2014).
5 Kamimoto, K. et al. Heterogeneity and stochastic growth regulation of biliary epithelial cells dictate dynamic epithelial tissue remodeling. Elife 5, e15034 (2016).
6 Tanimizu, N. et al. Progressive induction of hepatocyte progenitor cells in chronically injured liver. Sci Rep 7, 39990 (2017).
7 Zong, Y. et al. Notch signaling controls liver development by regulating biliary differentiation. Development 136, 1727-1739 (2009).
8 Sparks, E. E., Huppert, K. A., Brown, M. A., Washington, M. K. & Huppert, S. S. Notch signaling regulates formation of the three-dimensional architecture of intrahepatic bile ducts in mice. Hepatology 51, 1391-1400 (2010).
9 Jeliazkova, P. et al. Canonical Notch2 signaling determines biliary cell fates of embryonic hepatoblasts and adult hepatocytes independent of Hes1. Hepatology 57, 2469-2479 (2013).
10 Thorel, F. et al. Conversion of adult pancreatic alpha-cells to beta-cells after extreme beta-cell loss. Nature 464, 1149-1154 (2010).
11 Stange, D. E. et al. Differentiated Troy+ chief cells act as reserve stem cells to generate all lineages of the stomach epithelium. Cell 155, 357-368 (2013).
12 Jain, R. et al. Plasticity of Hopx(+) type I alveolar cells to regenerate type II cells in the lung. Nat Commun 6, 6727 (2015).
13 Tetteh, P. W. et al. Replacement of lost Lgr5-positive stem cells through plasticity of their enterocyte-lineage daughters. Cell Stem Cell 18, 203-213 (2016).
14 Van Eyken, P., Sciot, R. & Desmet, V. J. A cytokeratin immunohistochemical study of cholestatic liver disease: evidence that hepatocytes can express ‘bile duct-type’ cytokeratins. Histopathology 15, 125-135 (1989).
15 Libbrecht, L. et al. Peripheral bile duct paucity and cholestasis in the liver of a patient with Alagille syndrome: further evidence supporting a lack of postnatal bile duct branching and elongation. Am J Surg Pathol 29, 820-826 (2005).
16 Ernst, L. M., Spinner, N. B., Piccoli, D. A., Mauger, J. & Russo, P. Interlobular bile duct loss in pediatric cholestatic disease is associated with aberrant cytokeratin 7 expression by hepatocytes. Pediatr Dev Pathol 10, 383-390 (2007).
17 Fabris, L. et al. Analysis of liver repair mechanisms in Alagille syndrome and biliary atresia reveals a role for notch signaling. Am J Pathol 171, 641-653 (2007).
18 Michalopoulos, G. K., Barua, L. & Bowen, W. C. Transdifferentiation of rat hepatocytes into biliary cells after bile duct ligation and toxic biliary injury. Hepatology 41, 535-544 (2005).
19 Yanger, K. et al. Robust cellular reprogramming occurs spontaneously during liver regeneration. Genes Dev 27, 719-724 (2013).
20 Tanimizu, N., Nishikawa, Y., Ichinohe, N., Akiyama, H. & Mitaka, T. Sry HMG box protein 9-positive (Sox9+) epithelial cell adhesion molecule-negative (EpCAM−) biphenotypic cells derived from hepatocytes are involved in mouse liver regeneration. J Biol Chem 289, 7589-7598 (2014).
21 Nagahama, Y. et al. Contributions of hepatocytes and bile ductular cells in ductular reactions and remodeling of the biliary system after chronic liver injury. Am J Pathol 184, 3001-3012 (2014).
22 Li, L. et al. Alagille syndrome is caused by mutations in human Jagged1, which encodes a ligand for Notch1. Nat Genet 16, 243-251 (1997).
23 Oda, T. et al. Mutations in the human Jagged1 gene are responsible for Alagille syndrome. Nat Genet 16, 235-242 (1997).
24 McDaniell, R. et al. NOTCH2 mutations cause Alagille syndrome, a heterogeneous disorder of the notch signaling pathway. Am J Hum Genet 79, 169-173 (2006).
25 Poncy, A. et al. Transcription factors SOX4 and SOX9 cooperatively control development of bile ducts. Dev Biol 404, 136-148 (2015).
26 Walter, T. J., Vanderpool, C., Cast, A. E. & Huppert, S. S. Intrahepatic bile duct regeneration in mice does not require Hnf6 or Notch signaling through Rbpj. Am J Pathol 184, 1479-1488 (2014).
27 Gong, A. Y. et al. Somatostatin stimulates ductal bile absorption and inhibits ductal bile secretion in mice via SSTR2 on cholangiocytes. Am J Physiol Cell Physiol 284, C1205-1214 (2003).
28 Li, B. et al. Adult mouse liver contains two distinct populations of cholangiocytes. Stem Cell Reports 9, 478-489 (2017).
29 Peng, L. et al. RNA sequencing reveals dynamic changes of mRNA abundance of cytochromes P450 and their alternative transcripts during mouse liver development. Drug Metab Dispos 40, 1198-1209 (2012).
30 Rezvani, M. et al. In vivo hepatic reprogramming of myofibroblasts with AAV vectors as a therapeutic strategy for liver fibrosis. Cell Stem Cell 18, 809-816 (2016).
31 Fiorotto, R. et al. Notch signaling regulates tubular morphogenesis during repair from biliary damage in mice. J Hepatol 59, 124-130 (2013).
32 Clotman, F. et al. Control of liver cell fate decision by a gradient of TGF beta signaling modulated by Onecut transcription factors. Genes Dev 19, 1849-1854 (2005).
33 Clotman, F. et al. The onecut transcription factor HNF6 is required for normal development of the biliary tract. Development 129, 1819-1828 (2002).
34 Plumb-Rudewiez, N. et al. Transcription factor HNF-6/OC-1 inhibits the stimulation of the HNF-3alpha/Foxa1 gene by TGF-beta in mouse liver. Hepatology 40, 1266-1274 (2004).
35 Tanimizu, N. et al. Intrahepatic bile ducts are developed through formation of homogeneous continuous luminal network and its dynamic rearrangement in mice. Hepatology 64, 175-188 (2016).
36 Rougemont, A. L. et al. Bile ducts in regenerative liver nodules of Alagille patients are not the result of genetic mosaicism. J Pediatr Gastroenterol Nutr 61, 91-93 (2015).
37 Raven, A. et al. Cholangiocytes act as facultative liver stem cells during impaired hepatocyte regeneration. Nature 547, 350-354 (2017).
38 Mu, X. et al. Epithelial transforming growth factor-beta signaling does not contribute to liver fibrosis but protects mice from cholangiocarcinoma. Gastroenterology 150, 720-733 (2016).
39 Luche, H., Weber, O., Nageswara Rao, T., Blum, C. & Fehling, H. J. Faithful activation of an extra-bright red fluorescent protein in “knock-in” Cre-reporter mice ideally suited for lineage tracing studies. Eur J Immunol 37, 43-53 (2007).
40 Yamamoto, M. et al. A multifunctional reporter mouse line for Cre- and FLP-dependent lineage analysis. Genesis 47, 107-114 (2009).
41 Lakso, M. et al. Efficient in vivo manipulation of mouse genomic sequences at the zygote stage. Proc Natl Acad Sci USA 93, 5860-5865 (1996).
42 Snippert, H. J. et al. Intestinal crypt homeostasis results from neutral competition between symmetrically dividing Lgr5 stem cells. Cell 143, 134-144 (2010).
43 Leveen, P. et al. Induced disruption of the transforming growth factor beta type II receptor gene in mice causes a lethal inflammatory disorder that is transplantable. Blood 100, 560-568 (2002).
44 Madisen, L. et al. A robust and high-throughput Cre reporting and characterization system for the whole mouse brain. Nat Neurosci 13, 133-140 (2010).
45 Shinkai, Y. et al. RAG-2-deficient mice lack mature lymphocytes owing to inability to initiate V(D)J rearrangement. Cell 68, 855-867 (1992).
46 Cao, X. et al. Defective lymphoid development in mice lacking expression of the common cytokine receptor gamma chain. Immunity 2, 223-238 (1995).
47 Malato, Y. et al. Fate tracing of mature hepatocytes in mouse liver homeostasis and regeneration. J Clin Invest 121, 4850-4860 (2011).
48 Raymond, C. S. & Soriano, P. High-efficiency FLP and PhiC31 site-specific recombination in mammalian cells. PLoS One 2, e162 (2007).
49 Wieser, R., Wrana, J. L. & Massague, J. GS domain mutations that constitutively activate T beta R-I, the downstream signaling component in the TGF-beta receptor complex. EMBO J 14, 2199-2208 (1995).
50 Schindelin, J. et al. Fiji: an open-source platform for biological-image analysis. Nat Methods 9, 676-682 (2012).
51 Dorrell, C. et al. Prospective isolation of a bipotential clonogenic liver progenitor cell in adult mice. Genes Dev 25, 1193-1203 (2011).
52 Chen, Z., Wang, M., Xiang, Q., Sun, Z. & Zhang, R. The lectin of Dolichos biflorus agglutinin recognises glycan epitopes on the surface of a subset of cardiac progenitor cells. Cell Biol Int 37, 1238-1245 (2013).
53 Orellana, D. et al. Calmodulin controls liver proliferation via interactions with C/EBPbeta-LAP and C/EBPbeta-LIP. J Biol Chem 285, 23444-23456 (2010).
54 Siggs, O. M., Schnabl, B., Webb, B. & Beutler, B. X-linked cholestasis in mouse due to mutations of the P4-ATPase ATP11C. Proc Natl Acad Sci USA 108, 7890-7895 (2011).
55 Thakurdas, S. M. et al. Jagged1 heterozygosity in mice results in a congenital cholangiopathy which is reversed by concomitant deletion of one copy of Poglut1 (Rumi). Hepatology 63, 550-565 (2016).

Each of the references cited herein is hereby incorporated by reference in its entirety, or in relevant part, as would be apparent from the context of the citation.

The disclosed subject matter has been described with reference to various specific embodiments and techniques. It should be understood, however, that many variations and modifications may be made while remaining within the spirit and scope of the disclosed subject matter.

What is claimed is:

1. A method of forming a bile duct comprising introducing at least one expressible coding region of Transforming Growth Factor β Type I Receptor (TGFBR1), Transforming Growth Factor β Type II Receptor (TGFBR2), SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains into hepatocytes of a patient with a cholestatic disease or injury under conditions where the expression level of the coding region is greater than the wild-type level of expression, thereby inducing transdifferentiation of hepatocytes into mature cholangiocytes that form at least one bile duct.

2. The method of claim 1 wherein the coding region encodes TGFBR1.

3. The method of claim 1 wherein the patient has a cholestatic liver injury.

4. The method of claim 1 wherein the bile duct contributes to bile drainage.

5. The method of claim 1 wherein the bile duct is formed in the absence of Notch signaling.

6. The method of claim 2 wherein the TGFBR1 coding region is a constitutive allele.

7. The method of claim 2 wherein the TGFBR1 is under the expression control of a constitutive promoter.

8. The method of claim 7 wherein the constitutive promoter is the Elongation Factor 1α (EF1α) promoter.

9. The method of claim 2 wherein the TGFBR1 coding region is borne by a vector.

10. The method of claim 9 wherein the vector is a viral vector.

11. The method of claim 10 wherein the viral vector is Adeno-Associated Virus serotype 8.

12. The method of claim 3 wherein the injury results from a disease.

13. The method of claim 12 wherein the disease is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia.

14. The method of claim 13 wherein the disease is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, or trisomy 21.

15. The method of claim 14 wherein the disease is human Alagille syndrome (ALGS) and wherein the expressible coding region introduced into hepatocytes of the patient encodes JAG1, JAG2, DLL1, DLL3, DLL4, NOTCH1, NOTCH2, NOTCH3, NOTCH4 or any one or more of the respective NOTCH intracellular domains.

15. The method of claim 13 wherein the disease is primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, infections (CMV infection, reovirus type 3 infection, rubella infection, hepatitis C infection, hepatitis B infection, Epstein-Barr Virus infection; microbe infection, sarcoidosis, or idiopathic adulthood ductopenia.

16. The method of claim 14 wherein the disease is human Alagille syndrome (ALGS), primary biliary cholangitis, or primary sclerosing cholangitis.

17. The method of claim 1 wherein the expressible coding region of Transforming Growth Factor β Type I Receptor (TGFBR1), Transforming Growth Factor β Type II Receptor (TGFBR2), SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is introduced into the hepatocytes ex vivo.

18. The method of claim 17 wherein the coding region encodes TGFBR1.

19. The method of claim 1 wherein the expressible coding region of Transforming Growth Factor β Type I Receptor (TGFBR1), Transforming Growth Factor β Type II Receptor (TGFBR2), SMAD3, SMAD1, SMAD2, SMAD5, SMAD8/9, JAG1, JAG2, DLL1, DLL3, DLL4, NOTCH1, NOTCH2, NOTCH3, NOTCH4 or the respective NOTCH intracellular domains is introduced into the hepatocytes in vivo.

20. The method of claim 19 wherein the coding region encodes TGFBR1.

21. A method of treating a liver disease or injury in a patient by administering a therapeutically effective amount of a compound that induces increased activity of at least one protein effector in hepatocytes of the patient, wherein the protein effector is
(a) exogenously administered TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains;
(b) an mRNA encoding TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains; or
(c) endogenously expressed TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, NOTCH4 or any one or more of the respective NOTCH intracellular domains, wherein the endogenous expression is induced by a small activating RNA.

22. The method of claim 21 wherein the hepatocyte is a wild-type hepatocyte.

23. The method of claim 21 wherein the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia.

24. The method of claim 21 wherein the cholestatic disease or injury is human Alagille syndrome.

25. The method of claim 21 wherein the hepatocyte is a syngeneic hepatocyte.

26. The method of claim 21 wherein the hepatocyte is an autologous hepatocyte.

27. A method of inducing increased expression of an endogenous TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, or NOTCH4 expression control element, or gene, comprising administering a vector comprising a coding region for a guide RNA targeting the expression control region of TGFBR1, TGFBR2, SMAD3, SMAD1, SMAD2, SMAD5, SMAD8/9, JAG1, JAG2, DLL1, DLL3, DLL4, NOTCH1, NOTCH2, NOTCH3, or NOTCH4 and a coding region for a fusion of deactivated Cas9 (dCas9) and at least one transcriptional activator.

28. The method of claim 27 wherein the endogenous coding region, expression control element or gene is a JAG1 or NOTCH2 coding region, expression control element or gene.

29. The method of claim 27 wherein the transcriptional activator is VP64, p65, or Rta.

30. The method of claim 27 wherein the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia.

31. The method of claim 30 wherein the cholestatic disease or injury is human Alagille syndrome.

32. A method for correcting a mutated endogenous allele of a JAG1 or a NOTCH2 coding region, expression control element, or gene, comprising administering a vector comprising a coding region for a zinc finger nuclease (ZNF), a coding region for a transcription activator-like effector nuclease (TALEN), or coding regions for a guide RNA and Cas9 to a hepatocyte of a patient with a cholestatic disease or injury, thereby correcting the coding region of the mutated endogenous allele to treat the cholestatic disease or injury.

33. The method of claim 32 wherein the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia.

34. The method of claim 32, wherein the cholestatic disease or injury is human Alagille syndrome.

35. A method of forming at least one bile duct comprising transplanting an effective amount of a hepatocyte not engineered to overexpress a gene product to a patient with a cholestatic disease or injury, thereby inducing formation of at least one bile duct.

36. The method of claim 35 wherein the hepatocyte is a wild-type hepatocyte.

37. The method of claim 35 wherein the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia.

38. The method of claim 35 wherein the cholestatic disease or injury is human Alagille syndrome, primary biliary cholangitis, or primary sclerosing cholangitis.

39. The method of claim 35 wherein the hepatocyte is a syngeneic hepatocyte.

40. The method of claim 35 wherein the hepatocyte is an autologous hepatocyte.

41. The method of claim 39 or claim 40 wherein the patient has a mutant JAG1 or NOTCH2 allele, wherein the mutant allele is corrected by introducing a vector comprising a zinc finger nuclease (ZFN), a coding region for a transcription activator-like effector nuclease (TALEN), or coding regions for a guide RNA and Cas9 into a hepatocyte of a patient with a cholestatic disease or injury, wherein the vector is introduced into the hepatocyte in vitro, thereby correcting the coding region of the mutated endogenous allele to treat the cholestatic disease or injury.

42. The method of claim 39 or claim 40 wherein the patient has Alagille syndrome.

43. The method of claim 42 wherein the expression of an endogenous normal allele of JAG1 or NOTCH2 is induced by introducing in vitro a vector comprising a coding region for a guide RNA targeting the expression control region of the JAG1 or NOTCH2 allele, and a coding region for a fusion of deactivated Cas9 (dCas9) and at least one transcriptional activator into a hepatocyte of a patient with a cholestatic disease or injury.

44. A method of treating a cholestatic disease or injury comprising administering an effective amount of a hepatocyte not engineered to overexpress a gene product to a patient, thereby treating the cholestatic disease or injury.

45. The method of claim 44 wherein the hepatocyte is a wild-type hepatocyte.

46. The method of claim 44 wherein the cholestatic disease or injury is human Alagille syndrome (ALGS), biliary atresia, cystic fibrosis, alpha-1 antitrypsin deficiency, progressive familial intrahepatic cholestasis, arthrogryposis-renal dysfunction-cholestasis syndrome, trihydroxycoprostanic acidemia, trisomy 17, trisomy 18, trisomy 21, primary biliary cholangitis, primary sclerosing cholangitis, autoimmune hepatitis, acute rejection of liver transplant, chronic rejection of liver transplant, liver transplant ischemia, bone marrow transplant-induced chronic graft-versus-host disease, Hodgkin lymphoma, Langerhans cell histiocytosis, macrophage activation syndrome, Cytomegalovirus (CMV) infection, reovirus type 3 infection, rubella infection, hepatitis C infection, hepatitis B infection, Epstein-Barr Virus infection; microbe infection, sarcoidosis, or idiopathic adulthood ductopenia.

47. The method of claim 44 wherein the cholestatic disease or injury is human Alagille syndrome, primary biliary cholangitis, or primary sclerosing cholangitis.

48. The method of claim 44 wherein the hepatocyte is a syngeneic hepatocyte.

49. The method of claim 44 wherein the hepatocyte is an autologous hepatocyte.

50. The method of claim 48 or claim 49 wherein the patient has a mutant JAG1 or NOTCH2 allele, wherein the mutant allele is corrected by introducing a vector comprising a zinc finger nuclease (ZFN), a coding region for a transcription activator-like effector nuclease (TALEN), or coding regions for a guide RNA and Cas9 into a hepatocyte of a patient with a cholestatic disease or injury, wherein the vector is introduced into the hepatocyte in vitro, thereby correcting the coding region of the mutated endogenous allele to treat the cholestatic disease or injury.

51. The method of claim 48 or claim 49 wherein the patient has Alagille syndrome.

52. The method of claim 51 wherein the expression of an endogenous normal allele of JAG1 or NOTCH2 is induced by introducing in vitro a vector comprising a coding region for a guide RNA targeting the expression control region of the JAG1 or NOTCH2 allele, and a coding region for a fusion of deactivated Cas9 (dCas9) and at least one transcriptional activator into a hepatocyte of a patient with a cholestatic disease or injury.
